(19) United States
(12) Patent Application Publication
(10) Pub. No.: US 2026/0166225 A1
(43) Pub. Date: Jun. 18, 2026

(54) HEALTH TRACKING APPLICATIONS FOR SMART GLASSES

(71) Applicant: SoftEye, Inc., San Diego, CA (US)

(72) Inventors: Edwin Chongwoo Park, San Diego, CA (US); Te-Won Lee, San Diego, CA (US); DoYoung Lee, Seoul (KR); Ravishankar Sivalingam, San Jose, CA (US)

(73) Assignee: SoftEye, Inc., San Diego, CA (US)

(21) Appl. No.: 19/352,172

(22) Filed: Oct. 7, 2025

Related U.S. Application Data
(60) Provisional application No. 63/704,474, filed on Oct. 7, 2024.

(51) Int. Cl. A61M 5/172; G16H 20/17

(52) U.S. Cl. CPC A61M 5/1723; G16H 20/17; A61M 2205/3303; A61M 2205/3306; A61M 2205/3584; A61M 2205/52; A61M 2230/06; A61M 2230/201; A61M 2230/63

(57) ABSTRACT

Systems, computer programs, devices, and methods that enable coordination across multiple devices of the mobile ecosystem. In one embodiment, smart glasses detect when a user is about to eat food or take a drink and capture the consumable and portion. The data is recorded in a “morsel track” for health activity analysis. Low-fidelity captures provide preliminary recognition, while higher-fidelity captures are selectively invoked for definitive classification. Machine-learning logic generates predicted metabolic responses, such as real-time glucose trends, based on the recorded events. Predicted responses may dynamically adjust the operation of continuous glucose monitors, heart-rate sensors, or other biomedical devices. In some embodiments, the system triggers a pharmaceutical dispenser, such as an insulin pump, inhaler, or transdermal patch, to provide closed-loop therapeutic intervention in real time.

### PRIORITY

This application claims the benefit of priority to U.S. Provisional Patent Application Ser. No. 63/704,474 filed Oct. 7, 2024, and entitled “HEALTH TRACKING APPLICATIONS FOR SMART GLASSES”, incorporated by reference in its entirety.

### RELATED APPLICATIONS

This application is related to U.S. patent application Ser. No. 18/061,203 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/061,226 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/061,257 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/185,362 filed Mar. 16, 2023, and entitled “APPARATUS AND METHODS FOR AUGMENTING VISION WITH REGION-OF-INTEREST BASED PROCESSING”, U.S. patent application Ser. No. 18/185,364 filed Mar. 16, 2023, and entitled “APPARATUS AND METHODS FOR AUGMENTING VISION WITH REGION-OF-INTEREST BASED PROCESSING”, U.S. patent application Ser. No. 18/185,366 filed Mar. 16, 2023, and entitled “APPARATUS AND METHODS FOR AUGMENTING VISION WITH REGION-OF-INTEREST BASED PROCESSING”, U.S. patent application Ser. No. 18/316,181 filed May 11, 2023, and entitled “METHODS AND APPARATUS FOR SCALABLE PROCESSING”, U.S. patent application Ser. No. 18/316,214 filed May 11, 2023, and entitled “METHODS AND APPARATUS FOR SCALABLE PROCESSING”, U.S. patent application Ser. No. 18/316,206 filed May 11, 2023, and entitled “METHODS AND APPARATUS FOR SCALABLE PROCESSING”, U.S. patent application Ser. No. 18/316,214 filed May 11, 2023, and entitled “METHODS AND APPARATUS FOR SCALABLE PROCESSING”, U.S. patent application Ser. No. 18/316,218 filed May 11, 2023, and entitled “APPLICATIONS FOR ANAMORPHIC LENSES”, U.S. patent application Ser. No. 18/316,203 filed May 11, 2023, and entitled “APPLICATIONS FOR ANAMORPHIC LENSES”, U.S. patent application Ser. No. 18/316,221 filed May 11, 2023, and entitled “APPLICATIONS FOR ANAMORPHIC LENSES”, U.S. patent application Ser. No. 18/316,225 filed May 11, 2023, and entitled “APPLICATIONS FOR ANAMORPHIC LENSES”, U.S. patent application Ser. No. 18/745,027 filed Jun. 17, 2024, and entitled “NETWORK INFRASTRUCTURE FOR USER-SPECIFIC GENERATIVE INTELLIGENCE”, U.S. patent application Ser. No. 18/745,233 filed Jun. 17, 2024, and entitled “NETWORK INFRASTRUCTURE FOR USER-SPECIFIC GENERATIVE INTELLIGENCE”, U.S. patent application Ser. No. 18/745,353 filed Jun. 17, 2024, and entitled “NETWORK INFRASTRUCTURE FOR USER-SPECIFIC GENERATIVE INTELLIGENCE”, U.S. patent application Ser. No. 18/745,462 filed Jun. 17, 2024, and entitled “NETWORK INFRASTRUCTURE FOR USER-SPECIFIC GENERATIVE INTELLIGENCE”, U.S. patent application Ser. No. 18/745,779 filed Jun. 17, 2024, and entitled “NETWORK INFRASTRUCTURE FOR USER-SPECIFIC GENERATIVE INTELLIGENCE”, U.S. patent application Ser. No. 18/983,169 filed Dec. 16, 2024, and entitled “MACHINE-LEARNING ALGORITHMS FOR LOW-POWER APPLICATIONS”, U.S. patent application Ser. No. 18/983,220 filed Dec. 16, 2024, and entitled “MACHINE-LEARNING ALGORITHMS FOR LOW-POWER APPLICATIONS”, U.S. patent application Ser. No. 18/983,242 filed Dec. 16, 2024, and entitled “MACHINE-LEARNING ALGORITHMS FOR LOW-POWER APPLICATIONS”, U.S. patent application Ser. No. 18/983,261 filed Dec. 16, 2024, and entitled “MACHINE-LEARNING ALGORITHMS FOR LOW-POWER APPLICATIONS”, U.S. patent application Ser. No. 19/081,911 filed Mar. 17, 2025, and entitled “FOUNDATION MODEL PIPELINE FOR REAL-TIME EMBEDDED DEVICES”, U.S. patent application Ser. No. 19/081,924 filed Mar. 17, 2025, and entitled “FOUNDATION MODEL PIPELINE FOR REAL-TIME EMBEDDED DEVICES”, U.S. patent application Ser. No. 19/081,936 filed Mar. 17, 2025, and entitled “FOUNDATION MODEL PIPELINE FOR REAL-TIME EMBEDDED DEVICES”, and U.S. patent application Ser. No. 19/081,951 filed Mar. 17, 2025, and entitled “FOUNDATION MODEL PIPELINE FOR REAL-TIME EMBEDDED DEVICES”, each of which are incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### TECHNICAL FIELD

This disclosure relates generally to the field of health tracking applications. More particularly, the present disclosure relates to systems, computer programs, devices, and methods that enable coordination across multiple devices of the mobile ecosystem.

### DESCRIPTION OF RELATED TECHNOLOGY

A mobile device ecosystem is an interconnected network of hardware, software, and services that support mobile devices such as smart phones, smart watches, smart glasses, etc. It includes the devices themselves, mobile applications, and supporting infrastructure such as cloud services, wireless networks, and developer tools.

Smart glasses are wearable devices that resemble regular eyeglasses but integrate advanced technology, such as augmented reality (AR) displays, cameras, and sensors. These glasses allow users to view digital information overlaid onto their physical environment, interact with apps, and/or capture audio and video. Common uses include hands-free access to real-time data, navigation, communication, etc.

Health tracking sensors monitor various physiological metrics such as heart rate, activity levels, sleep patterns, blood oxygen levels, body temperature, blood sugar, etc. These sensors can provide real-time data on an individual's health and wellness to offer insights into overall well-being and/or detect early signs of potential health issues. They are widely used in fitness tracking, managing chronic conditions, and preventative healthcare.

### BRIEF DESCRIPTION OF THE DRAWINGS

FIG. 1 is a block diagram of a system configured to monitor and record a user's food consumption.

FIG. 2 is a graphical representation of a data structure configured to record and organize morsel events within a meal.

FIG. 3 depicts a morsel track and a real-time predicted interstitial glucose response, useful to illustrate prediction and response to physiological effects of food consumption.

FIG. 4 is a graphical representation of a scheme for adjusting continuous glucose monitoring based on predicted glucose response.

FIG. 5 is a graphical representation of a data structure configured to record multimodal health events for comprehensive metabolic tracking

FIG. 6 is a logical block diagram of one foundation model pipeline for real-time health tracking applications.

FIG. 7 is a logical block diagram of edge device.

FIG. 8 is a logical flow diagram for event-driven activity recognition and adaptive data capture.

### DETAILED DESCRIPTION

In the following detailed description, reference is made to the accompanying drawings. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without departing from the spirit or scope of the present disclosure. It should be noted that any discussion regarding “one embodiment”, “an embodiment”, “an exemplary embodiment”, and the like indicate that the embodiment described may include a particular feature, structure, or characteristic, and that such feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. The described operations may be performed in a different order than the described embodiments. Various additional operations may be performed and/or described operations may be omitted in additional embodiments.

### 1 Continuous Glucose Monitors and Food Tracking

A continuous glucose monitor (CGM) is a device used to continuously track glucose levels in a user's body, providing real-time data and trends throughout the day and night. During operation, a small sensor inserted under the skin, typically on the abdomen or arm, measures glucose levels in the interstitial fluid surrounding cells. The sensor relays this data to a smart phone (or other device) via a transmitter. A health tracking application running on the smart phone interprets the sensor readings and displays the information to the user. Compared to traditional finger-prick blood tests which “spot-check” the blood glucose at an instant in time, CGMs are more convenient and provide a complete record of the user's glucose metabolism. In some cases, CGMs may even alert the user to high levels (hyperglycemia) or low levels (hypoglycemia) to manage blood sugar more effectively.

Notably, CGMs measure glucose levels in interstitial fluid which is different than blood glucose levels. Interstitial glucose measurements lag behind blood glucose levels—particularly during rapid changes in glucose levels (eating and exercise). For example, when a user eats a meal, the time spent actually chewing and swallowing may be less than 10 minutes. 15-30 minutes later (after the meal), the real-time interstitial glucose starts to reflect the blood glucose increase. This is because the user's body must digest food and release the glucose into the blood before the interstitial glucose increases. Similarly, a user that is exercising is burning blood glucose rapidly and sweating (hydration affects accuracy); in these cases, the real-time interstitial glucose levels do not reflect the available blood glucose. In other words, even real-time interstitial glucose measurements are too late to affect the user's behavior.

Furthermore, sensor placement under the skin can cause discomfort or irritation and the body's healing response may affect sensor accuracy. Periodic replacement, re-insertion, and/or re-calibration may be needed to maintain accuracy. These factors affect the cost and convenience of use. CGMs may also give incorrect information and/or false alarms if the device is not properly maintained.

As a wholly separate tangent, “food tracking” refers to the practice of recording and monitoring the food and beverages a person consumes, typically to manage nutrition, calories, or dietary goals. Historically, a person might manually write down meals in a journal to track macronutrients (such as carbohydrates, proteins, and fats), micronutrients (like vitamins and minerals), and caloric intake. Visual inspection of food portions is imprecise and often subject to user bias; this may be further compounded when the recipe and/or food preparation is unknown (e.g., when eating out), etc.

More recently, some food tracking applications have attempted to incorporate pre-cataloged information and image logging to address the convenience and accuracy of food tracking. While phones are often more convenient than a paper journal, the process remains manual, inconvenient, and inaccurate. Some smart devices have attempted to incorporate video logging; video capture is extremely power intensive and the battery packs for such headsets are unsuitable for day-to-day use.

### 2 Intent-Based Health Tracking

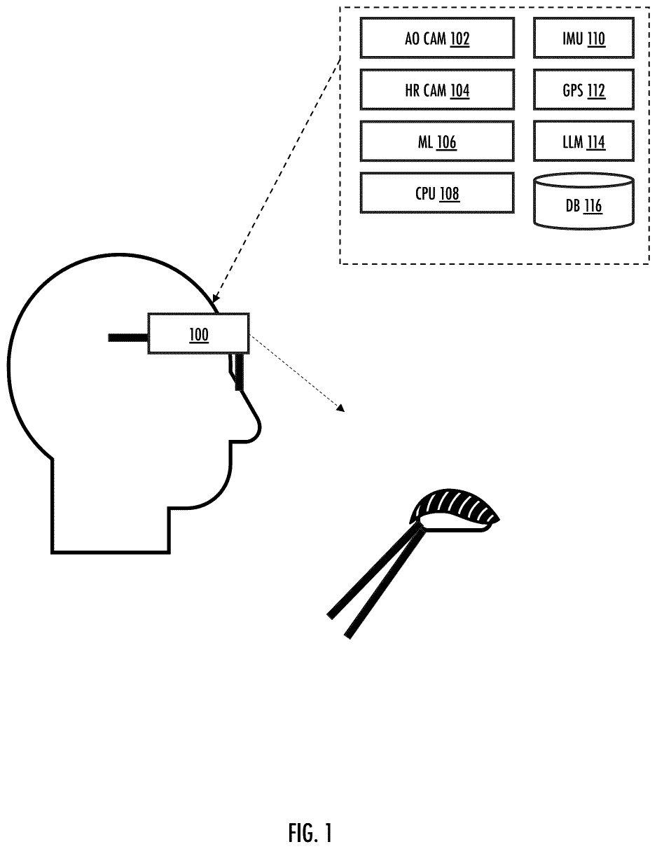

Various aspects of the present disclosure coordinate devices of the mobile ecosystem to enable intent-based health tracking. FIG. 1 illustrates a system 100 comprising components configured to monitor and record a user's food consumption. The system includes a low-resolution camera 102, a high-resolution camera 104, machine learning (ML) logic 106, a processor 108, an inertial measurement unit (IMU) 110, a global positioning system (GPS) 112, a large language model (LLM) 114, and a user database 116. The low-resolution camera 102 operates continuously at low power to monitor the user. The high-resolution camera 104 activates selectively to capture detailed images of food. The ML logic 106 analyzes captured data to determine portion size and food type. The processor 108 coordinates operation of the system components. The IMU 110 supports detection of head motions that signal eating activity. The GPS 112 records user location to provide contextual information. The LLM 114 queries external food databases to obtain nutrient information. The user database 116 stores time-stamped consumption data as a morsel track.

In one embodiment, smart glasses monitor eye intent to capture each “morsel” of an eating event. The morsel event may be triggered anytime a user quickly glances down in the area directly in front of the mouth with a hand positioned nearby (ostensibly holding food or a utensil with food). A morsel capture may include one or both of a low-power image (128×128, no IR illumination, long exposure) for low-power processing and a high-resolution image (640×480 or higher, IR illuminated, proper exposure) for subsequent image classification. The low-power captured image(s) of the event may be processed within onboard logic (e.g., low-power computer vision logic) to determine whether the user is eating a morsel or was a false alarm.

In one specific implementation, eye-tracking triggers a capture event whenever a user's gaze converges within an area angled approximately 30° to 60° below the eye's natural horizon line, spanning 60° across the centerline. A gaze fixation in this region for approximately 0.5 seconds to 0.7 seconds corresponds to a typical duration for a person to glance at an object before placing it in their mouth, but other threshold durations may be used with equal success (e.g., user configuration, etc.).

More broadly, the concepts may be readily extended to accommodate a variety of different user-specific, food-specific, and/or cultural eating habits. For example, some users may use shorter or longer glances before biting. Certain types of foods and/or beverages may be consumed differently (e.g., head tilt may be monitored for drinking, sipping from a straw, etc.). Some cultures eat with their hands, chopsticks, skewers, or other implements—these may affect hand positioning when taking bites, etc.

During operation, the system 100 monitors the user for a trigger event associated with consumption of a morsel. The low-resolution camera 102, operated at low power, performs continuous monitoring to detect visual cues of eating. The IMU 110 detects head motions that support recognition of the trigger event. The processor 108 coordinates the sensor data to reliably identify when a morsel event occurs.

Upon occurrence of the trigger event, the system enables the high-resolution camera 104 to capture a morsel event. The high-resolution camera 104 is powered only long enough to record a detailed image of the morsel. The processor 108 coordinates activation of the high-resolution camera 104 and ensures that only relevant events consume power and storage resources.

The system provides captured data to the ML logic 106 to identify the size and type of the morsel. The ML logic 106 processes the high-resolution image in conjunction with contextual data from the low-resolution camera 102. The processor 108 coordinates delivery of this data to ensure the ML logic 106 produces reliable identification. Generally, high-resolution images may be useful to provide accurate initial recognition, etc. (e.g., a specific menu item at a restaurant, a recipe commonly made at home, etc.); however, once a food item has been correctly identified, low-resolution images may be sufficient to track bite sizes and frequency since the user is taking bites from the same entrée.

The system then provides the morsel event information, together with relevant contextual information, to the LLM 114. The contextual information may include user location from the GPS 112 and temporal data from the processor 108. The LLM 114 queries external food databases to retrieve prorated nutrient data associated with the morsel. The processor 108 manages the integration of the morsel event information with the nutrient data.

Finally, the system stores the time-stamped prorated nutrient data in the user database 116 as a morsel track. The morsel track provides a historical record of food consumption. The processor 108 coordinates formatting of the data for storage, and the user database 116 maintains the morsel track for later retrieval and analysis.

As previously alluded to, journaling (written or image) often cannot capture significant details about a person's actual consumption. A person might order a dish, prepare a meal, measure a portion, etc., but fail to eat it all. For example, a user might share a dish with other people, have leftovers, etc. Similarly, a person might incorrectly estimate portion size e.g., eat a bowl of rice, not realizing that some bowls are bigger than others. Each morsel capture provides a granular accounting of how much a user ate and/or how quickly the user ate, etc. In other words, the morsel track records bites as they are taken, rather than a meal at a time.

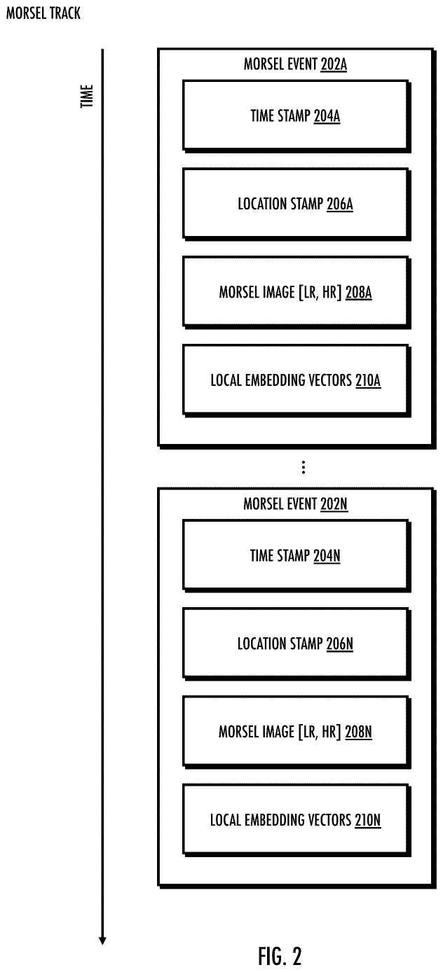

FIG. 2 illustrates a data structure configured to record and organize morsel events within a meal. The data structure includes morsel events 202A-202N, time stamps 204A-204N, location stamps 206A-206N, morsel images 208A-208N, and local embedding vectors 210A-210N. Each morsel event provides a record of consumption to enable analysis of nutrient intake as a function of ingestion rate. The time stamps record when each morsel event occurs. The location stamps may be used to identify the setting of the consumption event (e.g., a restaurant, home, friend's house, etc.). The morsel images include high-resolution images that enable food identification and portion size estimation, and low-resolution images that support triggering behavior and simplified processing. The local embedding vectors 210A-210N provide a mechanism to audit processing by machine learning and large language models.

In one embodiment, the morsel capture is stored as image data. For example, the low-resolution image and/or the high-resolution image may be stored with a time stamp within a “morsel track” data structure. Metadata information may include e.g., time of day, location, known food details, etc.

As previously alluded to, the smart glasses may include onboard logic to perform a visual analysis of the morsel and convert the information to another modality e.g., text, enumerated value, embedding vector, etc. For example, onboard computer vision analysis may identify an image of a spoonful of rice and chicken as “a spoonful of rice and chicken.” In some cases, the visual analysis may perform additional inferential steps e.g., “ 1/10th of a chicken and rice microwave dinner”, “15 grams of rice, 5 grams of chicken”, “15 grams of carbohydrates, 5 grams of protein”, etc.

Morsel events may be appended to the morsel track structure as they are gathered. In some cases, the morsel events may be further grouped into “meals”, “snacks”, etc. These groupings may more conveniently reflect the user's experience. Each morsel event includes a time stamp, and corresponding morsel images. A location stamp may be included with each morsel event and/or may be part of a broader meal grouping (e.g., a meal at a restaurant). The processor may coordinate insertion of these components into the morsel track. Local embedding vectors may be associated with the appended data to support later audit and verification of automated processing (e.g., audit ML identified foods, apportioning, etc. ; audit LLM interactions, etc.).

The morsel track may be used for subsequent analysis and audits. The morsel events provide temporal data that can be aligned with ingestion patterns. The location stamps allow contextual correlation with external food sources such as restaurant menus. The combined use of these components ensures that the morsel track provides a verifiable and analyzable record of food consumption.

Storing morsel data as images allows for subsequent post-processing and/or 3^{rd} party audits (e.g., a trained healthcare professional, etc.). However, image data may implicate privacy concerns—for example, images that capture unrelated information in the background (e.g., faces, device screens, etc.) may be undesirable for a variety of reasons. Some variants may use an additional layer of privacy protection logic to remove and/or obscure such information. For example, face detection logic may be used to identify faces to crop out/blur over. In one specific implementation, the privacy protection logic may be implemented on-device such that potentially private information is never exposed off the device.

Storing morsel data in formats other than images may also offer privacy protections. In other words, text-based morsel tracks greatly limit the amount of sensitive information that is transmitted off device. However, text-based morsel tracks may also limit the ability of a 3^{rd} party audit and/or subsequent post-processing.

In some embodiments, visual analysis may use contextual clues to infer morsel size. For example, liquid volumes may be inferred based on the cup size, liquid-level before drinking, and/or the liquid-level remaining after the drink, etc. A morsel size may be estimated based on how many bites are used to consume a pre-portioned meal (e.g., a microwave dinner) or a known-sized unit (e.g., a slice of pizza, a chicken drumstick, etc.). In some cases, apportionment may not be limited to a single user's activity-for example, a couple sharing a slice of pie might apportion the pie according to how many bites of the total number of bites taken were attributed to the user. Still other techniques may be substituted with equal success.

User interactions, gestures, and region-of-interest (ROI) processing are more broadly discussed within U.S. patent application Ser. No. 18/061,203 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/061,226 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/061,257 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/185,362 filed Mar. 16, 2023, and entitled “APPARATUS AND METHODS FOR AUGMENTING VISION WITH REGION-OF-INTEREST BASED PROCESSING”, U.S. patent application Ser. No. 18/185,364 filed Mar. 16, 2023, and entitled “APPARATUS AND METHODS FOR AUGMENTING VISION WITH REGION-OF-INTEREST BASED PROCESSING”, and U.S. patent application Ser. No. 18/185,366 filed Mar. 16, 2023, and entitled “APPARATUS AND METHODS FOR AUGMENTING VISION WITH REGION-OF-INTEREST BASED PROCESSING”, previously incorporated by reference in their entireties.

In one embodiment, a morsel track is provided to a smart phone for aggregation with other modalities of health tracking data and multi-modality processing. In one implementation, the smart phone also obtains interstitial glucose information from a continuous glucose monitor (CGM). CGM data may be stored according to timestamps that are time-aligned to the morsel tracks. Conceptually, the historic record of morsel tracks in temporal relation to the interstitial glucose tracks may be used to infer a real-time predicted interstitial glucose response to newly consumed morsels. While the following discussion is presented with reference to a morsel track and an interstitial glucose track, the concepts may be broadly extended to, and/or incorporate, other forms of timestamped metabolic information (e.g., heart rate tracking, oxygen consumption (VO₂), blood glucose (e.g., from finger pricks or other sampling), etc.).

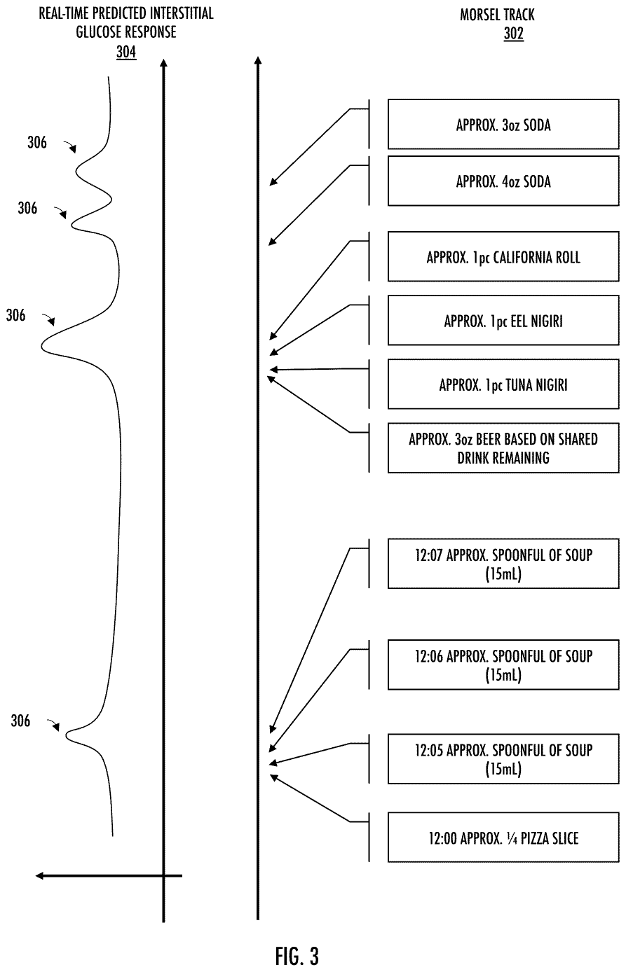

FIG. 3 depicts a morsel track 302, a real-time predicted interstitial glucose response 304, and peaks 306, useful to illustrate prediction and response to physiological effects of food consumption. The morsel track 302 provides a temporal record of consumption events. The real-time predicted interstitial glucose response 304 represents a machine learning-based prediction of the user's physiological response to ingestion of each morsel. The peaks 306 may trigger notifications to the user to slow eating, engage in physical activity to metabolize sugar, and/or initiate insulin delivery.

In one specific implementation, a machine-learning (ML) logic is trained on the historic timestamped morsel data and interstitial glucose data to generate a real-time predicted interstitial glucose response based on new morsel data. The ML logic may be trained in an offline mode (only historical data) or an online mode (historical data and/or live data). The trained model predicts the interstitial glucose response and monitors for trigger events as a function of ingestion. The morsel track 302 supplies sequential data of consumption, which serves as input to the predictive model. The model computes a real-time predicted interstitial glucose response 304 that reflects expected changes in blood sugar based on the ingested morsels. The system evaluates this response for identification of peaks 306 that represent a physiological threshold.

As a brief aside, the metabolic response may be influenced by a variety of factors that are difficult and/or impossible to directly measure. A non-exhaustive list may include e.g. the individual's innate biological mechanisms (e.g., organ health, gut biome, etc.). Other factors may roughly correspond to visually observable data e.g., the individual's style of eating (e.g., bite sizes, rate of eating, etc.), the sequence of eating (e.g., grains before vegetables before meat, etc.), the type of food (e.g., unrefined, processed, etc.), the timing of the eating (e.g., timing between bites, breaks between courses, etc.), etc. Image captures may have some ambiguity due to lookalikes (e.g., light beer and beer are visually indistinguishable, etc.) and/or image quality (e.g., volume is a 3-dimensional value estimated from a 2-dimensional image). Nonetheless, a user's eating habits over long periods tend to converge, resulting in good predictive outcomes.

ML logic may be trained to consider a variety of user-specific, event-specific, and/or food-specific factors. User-specific considerations may include e.g., size, age, ethnicity, gender, health conditions, heart rate, interstitial glucose level, food preferences, etc. Event-specific considerations may include e.g., time of day, time since last meal, time since last morsel, time since last exercise, home/restaurant dishes, etc. Food-specific considerations may include e.g. morsel macronutrients (carbohydrates, fats, proteins, etc.), morsel order, preparation (e.g., refined, processed, prepared, preserved, etc.), etc.

The system may respond to trigger events in real time. Upon detection of a peak 306, the system may notify the user to reduce ingestion rate. Alternatively, the system may recommend or initiate a physical activity, such as walking, to regulate glucose levels. In conditions where insulin therapy is required, the system may also trigger insulin delivery. These responses ensure that the user's physiological state is managed in relation to the ingestion record maintained by the morsel track 302.

In some cases, the real-time predicted interstitial glucose response may be further compared against the time-shifted real-time measured interstitial glucose response to improve model accuracy and/or provide the user with accuracy information. In one specific implementation, the real-time predicted interstitial glucose response and real-time measured interstitial glucose response may be cross-correlated to identify their respective similarity. As a brief aside, cross-correlation measures the similarity or relationship between two data sets as a function of a time shift applied to one of them. The two data sets are multiplied element-wise and summed at each time shift—the time shift with the largest value corresponds to the most likely time lag of the metabolic response. Other time series analysis may be substituted with equal success.

As a practical matter, existing food tracking techniques (e.g., manual journalling, etc.) have an error margin of 10-20%; once the ML logic provides predictions that match or exceed this accuracy, then the ML logic predictions may be used in conjunction with, or in place of, other health tracking data. For example, certain health care insurers may offer preferential rates and/or benefits to insured members that implement preventative measures (diet and exercise). Similarly, certain employers may benefit from reduced group rates and/or reimburse or subsidize health tracking in this manner. Health tracking applications may also extend to performance athletes and/or military personnel.

While ML-based prediction is described, other types of analytics may be substituted with equal success. For example, a look-up-based embodiment may perform a lookup through historic morsel records to identify a corresponding interstitial glucose response, etc. A crowd-sourced embodiment may assume an average metabolism, average morsel size, and/or average morsel composition to predict interstitial glucose response, etc.

Notably, individual metabolism changes at a relatively slow rate and does not require meal-to-meal granularity. Thus, some implementations may reduce a user's reliance on CGMs. For example, a user may implant a CGM for a “calibration” period. During this time, the user goes about their daily routine with their smart glasses generating real-time morsel tracks. Once the ML logic has trained itself to generate a real-time predicted interstitial glucose response from the real-time morsel tracks that matches the time-shifted real-time measured interstitial glucose response (meeting or exceeding the requisite accuracy), the CGM may be removed, and the ML logic may be used in open loop operation. Periodic re-calibration (using a CGM implant) may be performed at relatively infrequent intervals (several months apart e.g., 3 mo, 6 mo, etc.). CGM-like real-time predicted interstitial glucose data may be adequate for most health tracking applications and may be cheaper and more convenient than a CGM implant.

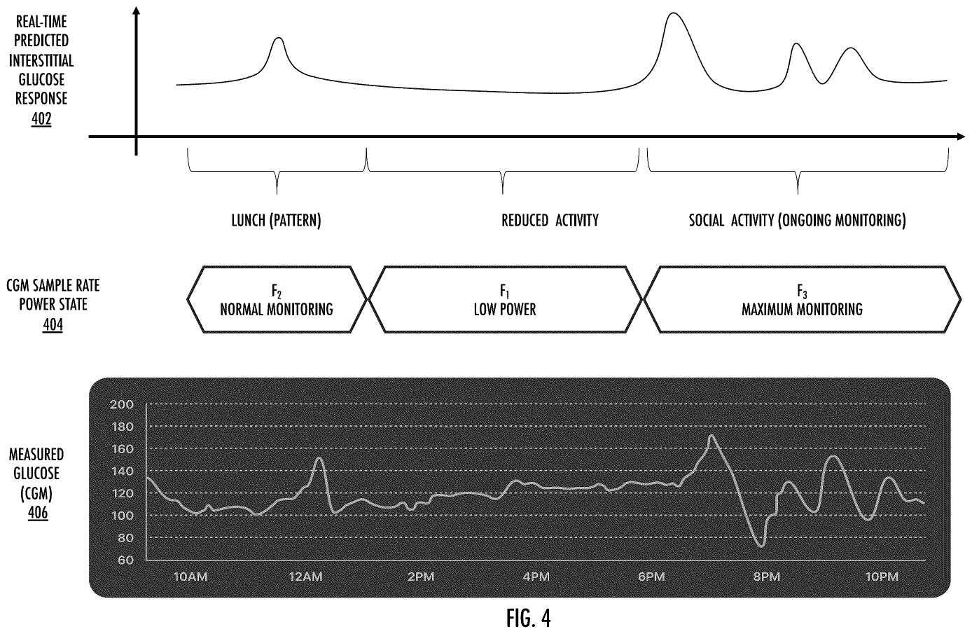

In some embodiments, the morsel track may be used to adjust the CGM's operation. FIG. 4 illustrates a scheme for adjusting continuous glucose monitoring based on predicted glucose response. Here, a real-time predicted interstitial glucose response 402 is generated by a machine learning model based on ingestion data. The CGM state 404 represents operational modes of the glucose monitor, with sampling frequencies that vary in accordance with user activity and predicted events. The measured glucose 406 represents the data generated by the CGM, recorded as a time-stamped track for analysis and training.

A machine learning model predicts the interstitial glucose response and monitors for trigger events as a function of ingestion. The predicted interstitial glucose response 402 represents expected blood sugar fluctuations derived from consumption records. The system evaluates these predictions to determine when a trigger event occurs that may be used to modify the CGM state 404.

For example, a CGM may operate at a relatively low sample rate and/or low sampling sensitivity to save power. Once the smart glasses capture an eating event, the smart glasses (or the smart phone) may instruct the CGM to increase its sampling rate and/or sampling sensitivity to catch the incipient interstitial glucose spike. Once the user has ended their meal (no morsels across a threshold time period), the CGM may be instructed to reduce sample rate and/or low sampling sensitivity to return to a lower power monitoring. Intelligent power management may extend implant battery life and/or further reduce the cost of operation. In other words, the trigger events may be used to adjust an operational state of a continuous glucose monitor. The CGM state 404 may transition to low sampling frequency during periods of low activity to conserve resources. The CGM state 404 may use normal sampling frequency when monitoring predictable events based on user patterns. For anomalous behaviors or uncertain events, the CGM state 404 may transition to high sampling frequency to maximize measurement accuracy.

The measured glucose levels may trade-off between granularity and power consumption. The measured glucose 406 may capture high-resolution data during unstable periods flagged by the predicted interstitial glucose response 402. During stable conditions, the CGM state 404 may reduce sampling frequency to extend device service life. The resulting measured glucose 406 is stored as a time-stamped track for subsequent analysis and model training.

For reference, a conventional CGM might collect interstitial glucose readings on a periodic basis (once every minute, once every five minutes, etc.). Interstitial glucose levels fall in the range of 40 mg/dL to 400 mg/dL; most CGMs have a sampling resolution of 8-12 bits. In addition to the sampling rate and granularity, CGMs also may include a small memory for buffering data between updates. A dynamically configured CGM might have a first power saving mode (a low sample rate, low sensitivity e.g., 8 bits every 5 minutes) and a second active mode (a high sample rate, high sensitivity e.g., 12 bits every 1 minute). Dynamic configuration provides the benefits of higher granularity and larger intervals between updates, with a long battery life.

While the following discussion is presented in the context of smart glasses in communication with a continuous glucose monitor (CGM) and/or a smart phone, the concepts may be broadly extended to a variety of other mobile devices for health tracking. Such devices may include, without limitation, monitoring devices such as e.g., activity trackers, heart rate trackers, sleep trackers. In some cases, the devices may also include medical devices and/or implants such as e.g., insulin pumps or other dosing apparatus, pacemakers, neurostimulators, etc.

A wide variety of factors may affect the user's metabolism. For example, sleep helps the body maintain balanced glucose levels. Insufficient sleep or disrupted sleep patterns can lead to higher blood sugar levels, as the body becomes less efficient at processing glucose—sleep tracking information may be used to adjust the ML model based on recent sleep and/or sleep habits. Similarly, recent exercise and frequent exercise also affect the body's immediate glucose metabolism and sensitivity to glucose. Activity trackers can be used to adjust the ML model based on exercise information.

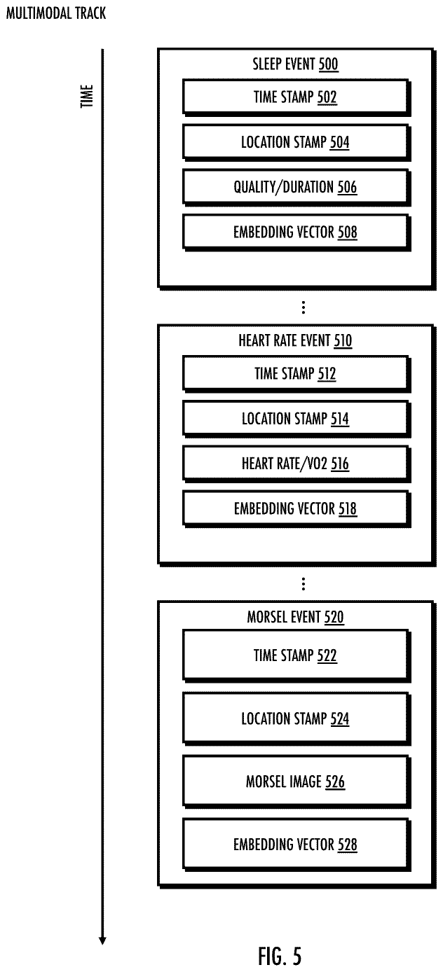

FIG. 5 illustrates a data structure configured to record multimodal health events for comprehensive metabolic tracking. The data structure includes a sleep event 500, a heart rate event 510, and a morsel event 520. The sleep event 500 records parameters within a sleep session, with associated time stamps 502, location stamps 504, sleep quality/duration 506, and embedding vectors 508. The heart rate event 510 records real-time heart activity, with associated time stamps 512, location stamps 514, heart rate/VO₂ data 516, and embedding vectors 518. The morsel event 520 records consumption events within a meal, with associated time stamps 522, location stamps 524, morsel images 526, and local embedding vectors 528. Collectively, these components form a multimodal account of metabolism.

Health events may be appended to the multimodal track structure. The sleep event 500, heart rate event 510, and morsel event 520 are inserted into the track with their corresponding time stamps 502, 512, 522 and location stamps 504, 514, 524. The processor may coordinate appending of these events to ensure temporal continuity and synchronization across modalities. Embedding vectors 508, 518, 528 may be associated with the appended data to support later audit and verification of automated processing.

The multimodal track may be used for subsequent analysis and audits. The sleep event 500 contributes data regarding sleep quality and duration 506, which may be correlated with metabolic efficiency. The heart rate event 510 contributes real-time physiological data, including heart rate and VO₂ data 516, that can be aligned with physical activity patterns. The morsel event 520 contributes nutrient intake data at ingestion resolution. When analyzed together, these events provide a multimodal account of metabolism that can be used for health assessment, prediction, and model validation.

In some variants, the mobile device ecosystem may even coordinate to prescribe and/or monitor user compliance. For example, a user that eats a large meal may be instructed to take a walk to reduce an impending blood glucose spike. Once on the walk, the user may receive a further notification that they've traveled far enough to mitigate the spike. Other interactions may be substituted with equal success, given the contents of the present disclosure.

Aggregation of multiple distinct modalities of instantaneous user context are more broadly discussed within U.S. patent application Ser. No. 18/745,027 filed Jun. 17, 2024, and entitled “NETWORK INFRASTRUCTURE FOR USER-SPECIFIC GENERATIVE INTELLIGENCE”, U.S. patent application Ser. No. 18/745,233 filed Jun. 17, 2024, and entitled “NETWORK INFRASTRUCTURE FOR USER-SPECIFIC GENERATIVE INTELLIGENCE”, U.S. patent application Ser. No. 18/745,353 filed Jun. 17, 2024, and entitled “NETWORK INFRASTRUCTURE FOR USER-SPECIFIC GENERATIVE INTELLIGENCE”, U.S. patent application Ser. No. 18/745,462 filed Jun. 17, 2024, and entitled “NETWORK INFRASTRUCTURE FOR USER-SPECIFIC GENERATIVE INTELLIGENCE”, U.S. patent application Ser. No. 18/745,779 filed Jun. 17, 2024, and entitled “NETWORK INFRASTRUCTURE FOR USER-SPECIFIC GENERATIVE INTELLIGENCE”, previously incorporated by reference in their entireties.

In addition to the foregoing aggregator functionality, the smart phone may additionally provide other post-capture processing (post-processing). Post-processing within the smart phone may enable/offload image processing and/or computer vision from the smart glasses. The smart phone may also have onboard computer vision logic, or access to cloud-based computer vision logic, to perform tasks at higher quality and/or best-effort (non-real-time). This may include e.g. segmentation, detection, localization, classification, and/or uniqueness determinations. The smart phone may also communicate health tracking information to others e.g., spouses, guardians, licensed medical professionals, friends, social network, etc.

Some embodiments may also provide a real-time display of the predicted interstitial glucose response. This may be particularly helpful for users to decide before they eat the next morsel, rather than via post-mortem analysis. Here, smart phone based displays allow a user to swipe through many different types of information, a smart glasses based display may provide an unobtrusive hands-free display of information the user is interested in.

Basic displays may only include numeric values and/or trend lines that correspond to the predicted interstitial glucose response. However, as previously alluded to, the human metabolic process is substantially more complex than just caloric intake and/or glucose level. In many cases, an individual may affect their metabolic efficiency by adjusting their eating habits (e.g., intervals between bites, the macronutrients of each bite, etc.). More complex displays may prescribe healthful eating advice (e.g., “wait another minute before having a bite”, “have a vegetable next”, etc.). In some cases, the displayed information may be implemented as graphical representations (e.g., a multi-colored bar graph showing recommended or actual amount of vegetables, meats, grains, and dairy, etc.). Various other modes of communicating information may be substituted with equal success.

While the foregoing discussion is presented in the context of a continuous glucose monitor (CGM), the concepts may be broadly extended to any biomedical device or implant which administers and/or monitors consumables that are metabolized by the body. As but one such example, current insulin pumps have attempted to directly coordinate with CGMs to administer insulin; however, insulin delivery is easily delayed due to the lag in interstitial glucose readings resulting in incorrect dosage. Both too little and too much insulin can cause adverse health outcomes. Embodiments that leverage morsel detection may estimate insulin dosage based on the ML model and/or real-time predicted interstitial glucose response to newly consumed morsels. In some variants, this may also be paired with a CGM implant (which is commonly used by diabetics) to supplement ML model with a real-time measured interstitial glucose response as well.

APPENDIX A incorporated herein by reference in its entirety describes usage scenarios for intent-based health tracking, useful in accordance with various aspects of the present disclosure.

APPENDIX B incorporated herein by reference in its entirety describes a mobile ecosystem that enables intent-based health tracking, useful in accordance with various aspects of the present disclosure.

### 3 Pipeline For Health Tracking

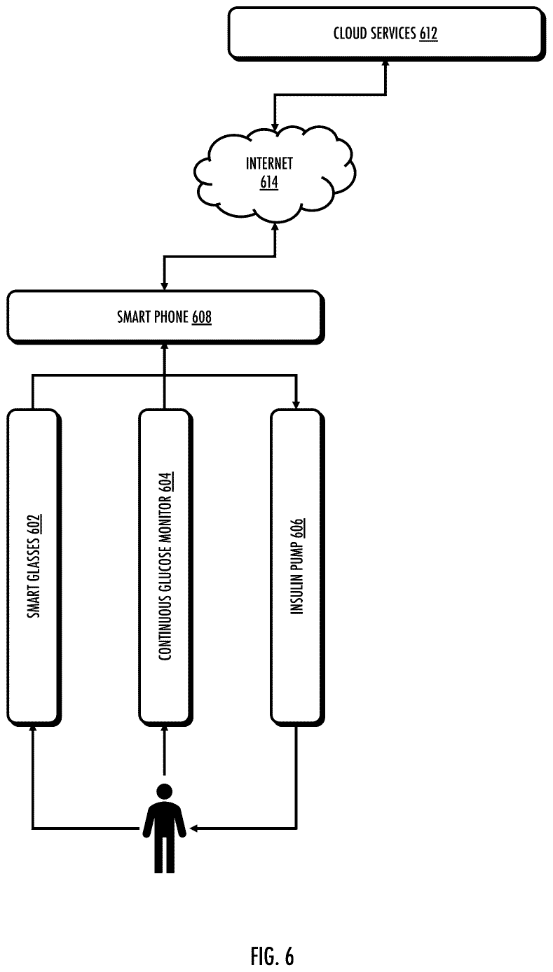

Various aspects of the present disclosure are now discussed with reference to a logical block diagram of one foundation model pipeline for real-time health tracking applications depicted within FIG. 6, useful in accordance with various aspects of the present disclosure. The illustrated pipeline is segmented into three (3) functional stages: edge capture stage, aggregation stage, and external resource stage.

As used herein, the term “pipeline” refers to a set of processing elements that process data in sequence, such that each processing element may also operate in parallel with the other processing elements. For example, a 3-stage pipeline may have first, second, and third processing elements that operate in parallel. During operation, the input of a second processing element includes at least the output of a first processing element, and the output of the second processing element is at least one input to a third processing element.

In one implementation, stages of the pipeline are handled within various entities of the mobile ecosystem. FIG. 6 illustrates a logical block diagram of a system for real-time health tracking. The system includes edge devices (smart glasses 602, a continuous glucose monitor (CGM) 604, an insulin pump 606), an aggregator device (a smart phone 608), and external resources (cloud services 612) accessible via the internet 614. As discussed in greater detail below, the edge devices manage real-time user interactions; e.g., the smart glasses 602 monitor user behavior for ingestion events, the CGM 604 monitors interstitial glucose levels as a proxy for the user's metabolism, the insulin pump 606 provides real-time pharmaceutical dosing in response to predicted or measured glucose levels, etc. The smart phone 608 functions as an aggregator that collects and coordinates data from the edge devices. The cloud services 612 provide remote computation and storage resources, and the internet 614 facilitates communication between the smart phone 608 and the cloud services 612.

The following examples are discussed in the context of edge devices that capture images and/or audio input from the user's environment, an aggregator device that aggregates edge capture data from multiple devices for cloud-based processing, and a cloud-based service that manages resource allocation and/or foundation model processing. More generally, however, artisans of ordinary skill in the related arts will readily appreciate that the functionalities described herein may be combined, divided, hybridized, and/or augmented within different entities. For example, a smart phone may have both edge functionality (e.g., capturing location information via GPS, etc.) as well as aggregator functionality (e.g., combining data streams from a connected smart glasses and smart watch). In another such example, a sufficiently capable smart phone may implement foundation model processing locally (rather than at a cloud service). Here, the smart phone may caption the instantaneous user context (or perform other forms of pre-processing) and/or aggregate the instantaneous user context for use with a local small LLM. The small LLM may then process the text data to identify what the user's attention is focused on. As yet another example, multiple distinct edge devices (e.g., smart glasses, smart phone, etc.) may communicate directly with a cloud service, which performs both aggregation as well as resource allocation, etc.

While the following discussion is presented in the context of a smart phone aggregator device that maintains a Bluetooth personal area network (PAN) with edge devices (smart glasses and smart watch), other types of devices and/or networks may be substituted with equal success. For example, a laptop, smart glasses, a smart watch, or smart car may provide network connectivity via hotspot, etc. Similarly, while present discussion is described in the context of Bluetooth, other networking technologies may be substituted with equal success. For instance, a smart phone may use Bluetooth/Wi-Fi ad hoc networking to connect to multiple devices of the user's mobile area network (e.g., smart glasses, smart watch, smart car, etc.).

### 3.1 Edge Capture

Edge devices refer to devices that are at the “edge” of the system—functionally, edge devices are used to capture the user's interactions and data about the environment and/or other instantaneous user context.

As a practical matter, edge devices may have a broad range of capability. For example, simple devices may capture data with sensors and pass the raw data to more sophisticated devices in the ecosystem. More sophisticated implementations may pre-process the instantaneous user context to detect user interest. Complex implementations may also aggregate data from other devices, implement localized processing, and/or even perform foundation model-type processing (e.g., large language models, large multimodal models, etc.). More broadly, any device that collects instantaneous user context may provide “edge device” functionality. For example, a smart phone may passively collect location information as part of its background tasks. Similarly, heart rate data may be collected from a smart watch, etc.

Edge devices may enforce localized control over data capture. For example, a user may enable or disable the cameras, microphones, and/or other sensors of their smart glasses for certain times of the day, certain activities, and/or certain locations. In some variants, the user may have the ability to provide default access settings and/or manually override default access settings.

While the following discussions are primarily discussed in the context of user-triggered data captures (which the user is aware of), edge devices may also receive and/or service capture requests from other entities (which the user may not be aware of). For example, an aggregator device may request a data capture either for its own operations, or on behalf of another entity (e.g., an LLM may need additional information about the user's context in order to provide a response). In some cases, the user may request/require notification for such accesses; other forms of access control may also be used (e.g., rule-based, etc.).

### 3.1.1 Implementation and Design Considerations

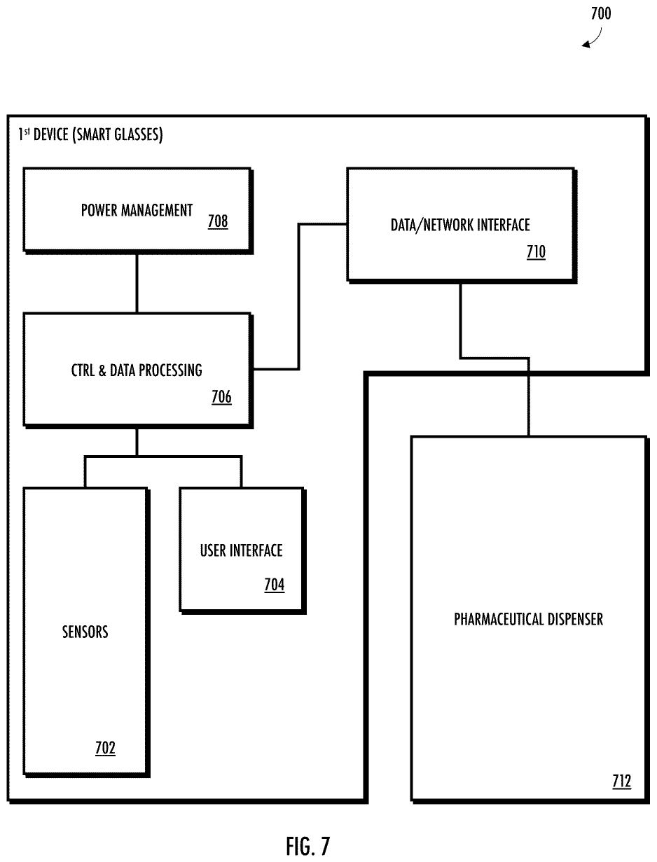

FIG. 7 is a logical block diagram of edge device 700. The edge device 700 includes: a sensor subsystem 702, a user interface subsystem 704, control and data processing logic 706, a power management subsystem 708, and a data/network interface 710. In some variants, the edge device 700 may additionally incorporate, or control, a pharmaceutical dispenser 712.

The sensor subsystem 702 captures data from the environment. The user interface subsystem 704 monitors the user for user interactions and renders data for user consumption. The control and data processing logic 706 obtains data generated by the user, other devices, and/or captured from the environment, to perform calculations and/or data manipulations. The resulting data may be stored, rendered to the user, transmitted to another party, or otherwise used by the edge device to carry out its tasks. The power management subsystem 708 supplies and controls power for the edge device components. The data/network interface 710 converts data for transmission to another device via removeable storage media or some other transmission medium. In some cases, the edge device may additionally include a physical frame that attaches the edge device to the user, freeing either one or both hands (hands-free operation).

The various logical subsystems described herein may be combined, divided, hybridized, and/or augmented within various physical components of a device. As but one such example, an inward-facing camera and outward-facing camera may be implemented as separate, or combined, physical assemblies. As another example, power management may be centralized within a single component or distributed among many different components; similarly, data processing logic may occur in multiple components of the edge device. More generally, the logical block diagram illustrates the various functional components of the edge device, which may be physically implemented in a variety of different manners.

Referring first to the sensor subsystem, a “sensor” refers to any electrical and/or mechanical structure that measures, and records, parameters of the physical environment as analog or digital data. Most consumer electronics devices incorporate multiple different modalities of sensor data; for example, visual data may be captured as images and/or video, audible data may be captured as audio waveforms (or their frequency representations), inertial measurements may be captured as quaternions, Euler angles, or other coordinate-based representations.

While the present disclosure is described in the context of audio data, visual data, and/or IMU data, artisans of ordinary skill in the related arts will readily appreciate that the raw data, metadata, and/or any derived data may be substituted with equal success. For example, an image may be provided along with metadata about the image (e.g., facial coordinates, object coordinates, depth maps, etc.). Post-processing may also yield derived data from raw image data; for example, a neural network may process an image and derive one or more activations.

In one embodiment, the sensor subsystem may include: one or more camera module(s), an audio module, an accelerometer/gyroscope/magnetometer (also referred to as an inertial measurement unit (IMU)), a display module (not shown), and/or Global Positioning System (GPS) system (not shown). Health tracking sensors may sense a variety of different physical activity, and their resulting metabolites, to infer a user's real-time metabolism. The following sections provide detailed descriptions of the individual components of the sensor subsystem.

A camera lens bends (distorts) light to focus on the camera sensor. The camera lens may focus, refract, and/or magnify light. It is made of transparent material such as glass or plastic and has at least one curved surface. When light passes through a camera lens, it is bent or refracted in a specific way, which can alter the direction, size, and/or clarity of the image that is formed.

A camera sensor senses light (luminance) via photoelectric sensors (e.g., photosites). A color filter array (CFA) filters light of a particular color; the CFA provides a color (chrominance) that is associated with each sensor. The combination of each luminance and chrominance value provides a mosaic of discrete red, green, blue value/positions, that may be “demosaiced” to recover a numeric tuple (RGB, CMYK, YUV, YCrCb, etc.) for each pixel of an image. Notably, most imaging formats are defined for the human visual spectrum; however, machine vision may use other variants of light. For example, a computer vision camera might operate on direct raw data from the image sensor with a RCCC (Red Clear Clear Clear) color filter array that provides a higher light intensity than the RGB color filter array used in media application cameras.

A camera sensor may be read using the readout logic. Conventional readout logic uses row enables and column reads to provide readouts in a sequential row-by-row manner. Historically, display devices were unaware of image capture but could optimize for their own raster-graphics scan line style of operation. Conventional data formats assign one dimension to be “rows” and another dimension to be “columns”; the row and column nomenclature is used by other components and/or devices to access data. Most (if not all) devices assume that scan lines are rows that run horizontally (left to right), and columns that run vertically (top to bottom), consistent with conventional raster-scan style operation.

A “digital image” is a two-dimensional array of pixels (or binned pixels). Virtually all imaging technologies are descended from (and inherit the assumptions of) raster-graphics displays which displayed images line-by-line. The aspect ratio of a digital image may be any number of pixels wide and high. However, images are generally assumed to be longer than they are tall (the rows are larger than columns).

During operation, the edge device may make use of multiple camera systems to assess user interactions and the physical environment. For example, smart glasses may have one or more outward-facing cameras to capture the user's environment. Multiple outward-facing cameras can be used to capture different fields-of-view and/or ranges. Cameras with a non-fixed/“zoom” lens may also change its focal length to capture multiple fields of view. For example, a medium range camera might have a horizontal field-of-view (FOV) of 70°-120° whereas long range cameras may use a FOV of 35°, or less, and have multiple aperture settings. In some cases, a “wide” FOV camera (so-called fisheye lenses provide between 120° and 195°) may be used to capture periphery information along two transverse axes. In some implementations, one or more anamorphic cameras may be used to capture a wide FOV in a first axis (major axis) and a medium range FOV in a second axis (minor axis). In addition, the smart glasses may have one or more inward-facing cameras to capture the user's interactions. Multiple cameras can be used to capture different views of the eyes for eye-tracking. In some implementations, one or more anamorphic cameras may be used to track eye movement. Other implementations may use normal FOV cameras that are stitched together or otherwise processed jointly.

More generally, however, any camera lens or set of camera lenses may be substituted with equal success for any of the foregoing tasks; including e.g., narrow field-of-view (10° to 90°) and/or stitched variants (e.g., 360° panoramas). While the foregoing techniques are described in the context of perceptible light, the techniques may be applied to other electromagnetic (EM) radiation capture and focus apparatus including without limitation: infrared, ultraviolet, and/or X-ray, etc.

The camera module(s) may include on-board image signal processing and/or neural network processing. On-board processing may be implemented within the same silicon or on a stacked silicon die (within the same package/module). Silicon and stacked variants reduce power consumption relative to discrete component alternatives that must be connected via external wiring, etc. Processing functionality is discussed further below.

The camera module(s) incorporates on-board logic to generate image analysis statistics and/or perform limited image analysis. As but one such example, the camera sensor may generate integral image data structures at varying scales. In some cases, the integral images may have reduced precision (e.g., only 8-bits, 12-bits, 16-bits, of precision). Notably, even at reduced precision, integral images may be used to calculate the sum of values in a patch of an image. This may enable lightweight computer vision algorithms that perform detection and/or recognition of objects, faces, text, etc.

More generally, a variety of applications may leverage preliminary image analysis statistics. For example, computer-assisted searches and/or other recognition algorithms, etc. are discussed in greater detail within U.S. patent application Ser. No. 18/185,362 filed Mar. 16, 2023, and entitled “APPARATUS AND METHODS FOR AUGMENTING VISION WITH REGION-OF-INTEREST BASED PROCESSING”, U.S. patent application Ser. No. 18/185,364 filed Mar. 16, 2023, and entitled “APPARATUS AND METHODS FOR AUGMENTING VISION WITH REGION-OF-INTEREST BASED PROCESSING”, and U.S. patent application Ser. No. 18/185,366 filed Mar. 16, 2023, and entitled “APPARATUS AND METHODS FOR AUGMENTING VISION WITH REGION-OF-INTEREST BASED PROCESSING”, previously incorporated by reference above.

Various embodiments of the present disclosure may additionally leverage improvements to scalable camera sensors and/or asymmetric camera lenses, discussed in greater detail within U.S. patent application Ser. No. 18/316,181 filed May 11, 2023, and entitled “METHODS AND APPARATUS FOR SCALABLE PROCESSING”, U.S. patent application Ser. No. 18/316,214 filed May 11, 2023, and entitled “METHODS AND APPARATUS FOR SCALABLE PROCESSING”, U.S. patent application Ser. No. 18/316,206 filed May 11, 2023, and entitled “METHODS AND APPARATUS FOR SCALABLE PROCESSING”, U.S. patent application Ser. No. 18/316,203 filed May 11, 2023, and entitled “APPLICATIONS FOR ANAMORPHIC LENSES”, U.S. patent application Ser. No. 18/316,218 filed May 11, 2023, and entitled “APPLICATIONS FOR ANAMORPHIC LENSES”, U.S. patent application Ser. No. 18/316,221 filed May 11, 2023, and entitled “APPLICATIONS FOR ANAMORPHIC LENSES”, U.S. patent application Ser. No. 18/316,225 filed May 11, 2023, and entitled “APPLICATIONS FOR ANAMORPHIC LENSES”, previously incorporated by reference above.

An audio module typically incorporates a microphone, speaker, and an audio codec. The microphone senses acoustic vibrations and converts the vibrations to an electrical signal (via a transducer, condenser, etc.). The electrical signal is provided to the audio codec, which samples the electrical signal and converts the time domain waveform to its frequency domain representation. Typically, additional filtering and noise reduction may be performed to compensate for microphone characteristics. The resulting audio waveform may be compressed for delivery via any number of audio data formats. To generate audible sound, the audio codec obtains audio data and decodes the data into an electrical signal. The electrical signal can be amplified and used to drive the speaker to generate acoustic waves.

Commodity audio codecs generally fall into speech codecs and full spectrum codecs. Full spectrum codecs use the modified discrete cosine transform (mDCT) and/or mel-frequency cepstral coefficients (MFCC) to represent the full audible spectrum. Speech codecs reduce coding complexity by leveraging the characteristics of the human auditory/speech system to mimic voice communications. Speech codecs often make significant trade-offs to preserve intelligibility, pleasantness, and/or data transmission considerations (robustness, latency, bandwidth, etc.).

An audio module may have any number of microphones and/or speakers. For example, multiple speakers may be used to generate stereo sound and multiple microphones may be used to capture stereo sound. More broadly, any number of individual microphones and/or speakers can be used to constructively and/or destructively combine acoustic waves (also referred to as beamforming). The audio module may include on-board audio processing and/or neural network processing to assist with voice analysis and synthesis.

The inertial measurement unit (IMU) may include one or more accelerometers, gyroscopes, and/or magnetometers. Typically, an accelerometer uses a damped mass and spring assembly to measure proper acceleration (i.e., acceleration in its own instantaneous rest frame). In many cases, accelerometers may have a variable frequency response. Most gyroscopes use a rotating mass to measure angular velocity; a MEMS (microelectromechanical) gyroscope may use a pendulum mass to achieve a similar effect by measuring the pendulum's perturbations. Most magnetometers use a ferromagnetic element to measure the vector and strength of a magnetic field; other magnetometers may rely on induced currents and/or pickup coils. The IMU uses the acceleration, angular velocity, and/or magnetic information to calculate quaternions that define the relative motion of an object in four-dimensional (4D) space. Quaternions can be efficiently computed to determine velocity (both head direction and speed).

More generally, however, any scheme for detecting user velocity (direction and speed) may be substituted with equal success for any of the foregoing tasks. Other useful information may include pedometer and/or compass measurements. While the foregoing techniques are described in the context of an inertial measurement unit (IMU) that provides quaternion vectors, artisans of ordinary skill in the related arts will readily appreciate that raw data (acceleration, rotation, magnetic field) and any of their derivatives may be substituted with equal success.

Global Positioning System (GPS) is a satellite-based radio navigation system that allows a user device to triangulate its location anywhere in the world. Each GPS satellite carries very stable atomic clocks that are synchronized with one another and with ground clocks. Any drift from time maintained on the ground is corrected daily. In the same manner, the satellite locations are known with great precision. The satellites continuously broadcast their current position. During operation, GPS receivers attempt to demodulate GPS satellite broadcasts. Since the speed of radio waves is constant and independent of the satellite speed, the time delay between when the satellite transmits a signal and the receiver receives it is proportional to the distance from the satellite to the receiver. Once received, a GPS receiver can triangulate its own four-dimensional position in spacetime based on data received from multiple GPS satellites. At a minimum, four satellites must be in view of the receiver for it to compute four unknown quantities (three position coordinates and the deviation of its own clock from satellite time). In so-called “assisted GPS” implementations, ephemeris data may be downloaded from cellular networks to reduce processing complexity (e.g., the receiver can reduce its search window). The IMU may include on-board telemetry processing and/or neural network processing to assist with telemetry analysis and synthesis.

A continuous glucose monitor (CGM) measures interstitial glucose levels as a proxy for the user's metabolism. Inputs include interstitial fluid glucose concentrations sampled by a subcutaneous sensor. Outputs include time-stamped glucose readings transmitted to the smart phone for synchronization with morsel events.

One implementation of the CGM uses an electrochemical sensor comprising a glucose oxidase enzyme layer and a microelectrode array. The sensor oxidizes glucose in the interstitial fluid, generating an electrical current proportional to glucose concentration. The raw current is digitized, filtered, and packaged as glucose readings before transmission.

CGM sensors may trade-off between sampling frequency and sensor lifetime. High-frequency sampling increases temporal resolution but accelerates battery depletion and enzyme degradation. Low-frequency sampling extends device life but reduces the granularity of glucose tracking. Adaptive sampling (e.g., based on ingestion events detected from smart glasses) balances accuracy with resource conservation.

A heart rate tracker monitors cardiovascular activity and provides time-stamped heart rate or related metrics to the aggregator. Inputs include optical or electrical physiological signals obtained from the user. Outputs include derived heart rate values, heart rate variability, and related indicators transmitted to the smart phone for synchronization with other modalities.

One implementation of the heart rate tracker uses photoplethysmography (PPG). A light-emitting diode projects light into the skin, and a photodiode measures variations in reflected light corresponding to blood volume changes. A digital signal processor filters the PPG signal to remove noise, extract periodic peaks, and compute beats per minute. Optional calculations of inter-beat intervals may be used for heart rate variability.

The heart rate tracker may trade-off between accuracy and power consumption. High sampling rates and multi-wavelength PPG improve accuracy, especially during motion, but drain battery life. Low sampling rates extend device life but may miss transient changes. An alternative mechanism is an electrocardiogram (ECG) sensor, which offers medical-grade accuracy but requires higher electrode contact quality and special handling. Hybrid designs may selectively activate ECG recording when anomalous PPG patterns are detected, balancing power use with accuracy.

VO₂ sensors quantify a user's oxygen consumption rate. Inputs include respiratory flow and gas composition signals, or surrogate signals such as heart rate, accelerometer vectors, GPS speed, and external power. Outputs include instantaneous VO₂ (mL·min⁻¹), mass-normalized VO₂ (mL·kg⁻¹·min⁻¹), rolling averages, estimated energy expenditure, and quality flags.

Typically, VO₂ sensors use breath-by-breath indirect calorimetry with a flow path, an O₂ sensor, and a CO₂ sensor. A differential pressure or turbine sensor measures inspiratory/expiratory flow to integrate tidal volume per breath. A galvanic or paramagnetic O₂ sensor and a non-dispersive infrared CO₂ sensor sample fractional concentrations with known response times; a delay-alignment routine phase-matches gas fractions to volumetric flow. Direct VO₂ measurement in this manner is generally too cumbersome for wearable applications; more recently however, wearables have been able to generate a VO₂-like estimations using the PPG based sensor. One implementation of the PPG-based VO₂ measurement subsystem uses a dual-wavelength PPG sensor in conjunction with an accelerometer. The internal mechanism acquires red and infrared PPG signals to compute oxygen saturation (SpO₂) and derive pulse rate and waveform morphology. The accelerometer provides cadence and motion intensity as contextual inputs. A regression model trained against indirect calorimetry datasets processes these inputs to estimate VO₂ in real time.

The PPG-based VO₂ measurement subsystem optimizes wearability and non-invasiveness at the expense of accuracy. The selected implementation avoids bulky respiratory gas-exchange systems but relies on correlative models that require calibration and may lose precision under high-intensity motion. An alternative implementation could use direct breath-by-breath gas analysis, which increases accuracy but reduces portability. Another alternative implementation could employ hybrid modeling, where periodic calibration sessions with indirect calorimetry improve long-term accuracy while maintaining wearable convenience.

Referring now to the user interface subsystem, the “user interface” refers to the physical and logical components of the edge device that interact with the human user. A “physical” user interface refers to electrical and/or mechanical devices that the user physically interacts with. An “augmented reality” user interface refers to a user interface that incorporates an artificial environment that has been overlaid on the user's physical environment. A “virtual reality” user interface refers to a user interface that is entirely constrained within a “virtualized” artificial environment. An “extended reality” user interface refers to any user interface that lies in the spectrum from physical user interfaces to virtual user interfaces.

The user interface subsystem may encompass the visual, audio, and tactile elements of the device that enable a user to interact with it. In addition to physical user interface devices that use physical buttons, switches, and/or sliders to register explicit user input, the user interface subsystem may also incorporate various components of the sensor subsystem to sense user interactions. For example, the user interface may include: a display module to present information, eye-tracking camera sensor(s) to monitor gaze fixation, hand-tracking camera sensor(s) to monitor for hand gestures, a speaker to provide audible information, and a microphone to capture voice commands, etc.

The display module is an output device for presentation of information in a visual form. Different display configurations may internalize or externalize the display components within the lens. For example, some implementations embed optics or waveguides within the lens and externalize the display as a nearby projector or micro-LEDs. As another such example, some implementations project images into the eyes.

The display module may be incorporated within the device as a display that is overlaps the user's visual field. Examples of such implementations may include so-called “heads up displays” (HUDs) that are integrated within the lenses, or projection/reflection type displays that use the lens components as a display area. Existing integrated display sizes are typically limited to the lens form factor, and thus resolutions may be smaller than handheld devices e.g., 640×320, 1280×640, 1980×1280, etc. For comparison, handheld device resolutions that exceed 2560×1280 are not unusual for smart phones, and tablets can often provide 4K UHD (3840×2160) or better. In some embodiments, the display module may be external to the glasses and remotely managed by the device (e.g., screen casting). For example, smart glasses can encode a video stream that is sent to a user's smart phone or tablet for display.

The display module may be used where smart glasses present and provide interaction with text, pictures, and/or AR/XR objects. For example, the AR/XR object may be a virtual keyboard and a virtual mouse. During such operation, the user may invoke a command (e.g., a hand gesture) that causes the smart glasses to present the virtual keyboard for typing by the user. The virtual keyboard is provided by presenting images on the smart glasses such that the user may type without contact to a physical object. One of ordinary skill in the art will appreciate that the virtual keyboard (and/or mouse) may be displayed as an overlay on a physical object, such as a desk, such that the user is technically touching a real-world object. However, input is measured by tracking user movements relative to the overlay, previous gesture position(s), etc. rather than receiving a signal from the touched object (e.g., as a conventional keyboard would).

The user interface subsystem may incorporate an “eye-tracking” camera to monitor for gaze fixation (a user interaction event) by tracking saccadic or microsaccadic eye movements. Eye-tracking embodiments may greatly simplify camera operation since the eye-tracking data is primarily captured for standby operation (discussed below). In addition, the smart glasses may incorporate “hand-tracking” or gesture-based inputs. Gesture-based inputs and user interactions are more broadly described within e.g., U.S. patent application Ser. No. 18/061,203 filed Dec. 2, 2022, and entitled “SYSTEMS, APPARATUS, AND METHODS FOR GESTURE-BASED AUGMENTED REALITY, EXTENDED REALITY”, U.S. patent application Ser. No. 18/061,226 filed Dec. 2, 2022, and entitled “SYSTEMS, APPARATUS, AND METHODS FOR GESTURE-BASED AUGMENTED REALITY, EXTENDED REALITY”, and U.S. patent application Ser. No. 18/061,257 filed Dec. 2, 2022, and entitled “SYSTEMS, APPARATUS, AND METHODS FOR GESTURE-BASED AUGMENTED REALITY, EXTENDED REALITY”, previously incorporated by reference in their entireties.

While the present discussion describes inward-facing and hand-tracking cameras, the techniques are broadly applicable to any outward-facing and inward-facing cameras. As used herein, the term “outward-facing” refers to cameras that capture the surroundings of a user and/or the user's relation relative to the surroundings. For example, a rear outward-facing camera could be used to capture the surroundings behind the user. Such configurations may be useful for gaming applications and/or simultaneous localization and mapping (SLAM-based) applications. As used herein, the term “inward-facing” refers to cameras that capture the user e.g., to infer user interactions, etc.

The user interface subsystem may incorporate microphones to collect the user's vocal instructions as well as the environmental sounds. As previously noted above, the audio module may include on-board audio processing and/or neural network processing to assist with voice analysis and synthesis.

The user interface subsystem may also incorporate speakers to reproduce audio waveforms. In some cases, the speakers may incorporate noise reduction technologies and/or active noise cancelling to cancel out external sounds, creating a quieter listening environment for the user. This may be particularly useful for sensory augmentation in noisy environments, etc.

Functionally, the data/network interface subsystem enables communication between devices. For example, the edge device may communicate with an aggregator device. In some cases, the edge device may also need to access remote data (accessed via an intermediary network). For example, a user may want to look up a menu from a QR code (which visually embeds a network URL) or store a captured picture to their social network, social network profile, etc. In some cases, the user may want to store data to removable media. These transactions may be handled by a data interface and/or a network interface.

The network interface may include both wired interfaces (e.g., Ethernet and USB) and/or wireless interfaces (e.g., cellular, local area network (LAN), personal area network (PAN)) to a communication network. As used herein, a “communication network” refers to an arrangement of logical nodes that enables data communication between endpoints (an endpoint is also a logical node). Each node of the communication network may be addressable by other nodes; typically, a unit of data (a data packet) may be traverse across multiple nodes in “hops” (a segment between two nodes). For example, smart glasses may directly connect, or indirectly tether to another device with access to, the Internet. “Tethering” also known as a “mobile hotspot” allows devices to share an internet connection with other devices. For example, a smart phone may use a second network interface to connect to the broader Internet (e.g., 5G/6G cellular); the smart phone may provide a mobile hotspot for a smart glasses device over a personal area network (PAN) interface (e.g., Bluetooth/Wi-Fi), etc.

The data interface may include one or more removeable media. Removeable media refers to a memory that may be attached/removed from the edge device. In some cases, the data interface may map (“mount”) the removable media to the edge device's internal memory resources to expand its operational memory.

Functionally, the pharmaceutical dispenser delivers doses of pharmacologically active substance (e.g., a chemical or biological substance used to diagnose, treat, and/or alleviate a health issue). The doses are administered in real-time or near-real time, in response to predicted or measured user activity. User health activity may include both their controlled behaviors (e.g., eating, sleeping, resting, etc.), as well as their uncontrolled behaviors (e.g., metabolism, etc.).

One implementation of the pharmaceutical dispenser is an insulin pump that delivers insulin based on a control API that is connected to e.g., the smart phone, smart glasses, etc. The control API may receive dosage amounts, times, which cause delivery of insulin via a subcutaneous cannula. The insulin pump may use a stepper motor that actuates a plunger in an insulin reservoir. The motor position is precisely controlled to deliver microliter-scale doses. A microcontroller receives dosage instructions, converts them to motor actuation signals, and verifies delivery through a feedback loop using position encoders. High-precision stepper motors provide reliable dosing but increase pump cost and power consumption. Simpler peristaltic mechanisms could reduce complexity and cost but at the expense of dosing granularity. Closed-loop verification ensures safety but adds processing overhead.

While the foregoing discussion is described within the context of insulin which is used by the user's body to metabolize sugar, pharmaceutical dispensing devices are used in a variety of medical applications including e.g., pain management and palliative care, cancer/heart disease, addiction, mental health, contraceptives, etc. The concepts described throughout may be readily used to monitor the user for activity that affects/indicates the metabolism of a pharmaceutical and trigger an automated release. For example, a patient that receives pain relief medications (opiods) may be monitored with eye tracking, heart rate, etc. to identify therapeutic use, diminishing efficacy, and/or symptoms predicting the onset of addiction. In other words, therapeutic use seeks to return user indications back to baseline (e.g., reductions from an elevated heart rate, return to normal eye movement and pupillary dilation) whereas abuse triggers deviations from baseline (e.g., increasing heart rate from baseline, frenetic eye movements and abnormal dilation, etc.).

Furthermore, while automated dispensing may be convenient, the concepts are broadly applicable to other user activities. Certain aspects of user health may have unpredictable timing and/or may be halted with preventative action and/or medication. For example, migraine headaches may have biological markers that predict onset. Early notification may allow a user to seek a safe refuge before onset. Similarly, certain users experience unpredictable menstrual cycles for a variety of reasons. Monitoring for predictive biological markers may be useful to detect and instruct a user to administer birth control, change behavior/environment, etc. Various other applications may be substituted with equal success.

The control and data subsystem controls the operation of a device and stores and processes data. Logically, the control and data subsystem may be subdivided into a “control path” and a “data path.” The data path is responsible for performing arithmetic and logic operations on data. The data path generally includes registers, arithmetic and logic unit (ALU), and other components that are needed to manipulate data. The data path also includes the memory and input/output (I/O) devices that are used to store and retrieve data. In contrast, the control path controls the flow of instructions and data through the subsystem. The control path usually includes a control unit, that manages a processing state machine (e.g., a program counter which keeps track of the current instruction being executed, instruction register which holds the current instruction being executed, etc.). During operation, the control path generates the signals that manipulate data path operation. The data path performs the necessary operations on the data, and the control path moves on to the next instruction, etc.

The control and data processing logic may include one or more of: a central processing unit (CPU), an image signal processor (ISP), one or more neural network processors (NPUs), and their corresponding non-transitory computer-readable media that store program instructions and/or data. In one exemplary embodiment, the control and data subsystem includes processing units that execute instructions stored in a non-transitory computer-readable medium (memory). More generally however, other forms of control and/or data may be substituted with equal success, including e.g., neural network processors, dedicated logic (field programmable gate arrays (FPGAs), application specific integrated circuits (ASICs)), and/or other software, firmware, and/or hardware implementations.

Different processor architectures attempt to optimize their designs for their most likely usages. More specialized logic can often result in much higher performance (e.g., by avoiding unnecessary operations, memory accesses, and/or conditional branching). For example, a general-purpose CPU may be primarily used to control device operation and/or perform tasks of arbitrary complexity/best-effort. CPU operations may include, without limitation: operating system (OS) functionality (power management, UX), memory management, gesture-specific tasks, etc. Typically, such CPUs are selected to have relatively short pipelining, longer words (e.g., 32-bit, 64-bit, and/or super-scalar words), and/or addressable space that can access both local cache memory and/or pages of system virtual memory. More directly, a CPU may often switch between tasks, and must account for branch disruption and/or arbitrary memory access.

In contrast, the image signal processor (ISP) performs many of the same tasks repeatedly over a well-defined data structure. Specifically, the ISP maps captured camera sensor data to a color space. ISP operations often include, without limitation: demosaicing, color correction, white balance, and/or autoexposure. Most of these actions may be done with scalar vector-matrix multiplication. Raw image data has a defined size and capture rate (for video) and the ISP operations are performed identically for each pixel; as a result, ISP designs are heavily pipelined (and seldom branch), may incorporate specialized vector-matrix logic, and often rely on reduced addressable space and other task-specific optimizations. ISP designs only need to keep up with the camera sensor output to stay within the real-time budget; thus, ISPs more often benefit from larger register/data structures and do not need parallelization.

In some cases, the device may include one or more neural network processors (NPUs). Unlike the Turing-based processor architectures, machine learning algorithms learn a task that is not explicitly described with instructions. In other words, machine learning algorithms seek to create inferences from patterns in data using e.g., statistical models and/or analysis. The inferences may then be used to formulate predicted outputs that can be compared to actual output to generate feedback. Each iteration of inference and feedback is used to improve the underlying statistical models. Since the task is accomplished through dynamic coefficient weighting rather than explicit instructions, machine learning algorithms can change their behavior over time to e.g., improve performance, change tasks, etc.

Conceptually, neural network processing uses a collection of small nodes to loosely model the biological behavior of neurons. Each node receives inputs, and generates output, based on a neuron model (usually a rectified linear unit (ReLU), or similar). The nodes are connected to one another at “edges”. Each node and edge are assigned a weight. Each processor node of a neural network combines its inputs according to a transfer function to generate the outputs. The set of weights can be configured to amplify or dampen the constituent components of its input data. The input-weight products are summed and then the sum is passed through a node's activation function, to determine the size and magnitude of the output data. “Activated” neurons (processor nodes) generate output “activations”. The activation may be fed to another node or result in an action on the environment. Coefficients may be iteratively updated with feedback to amplify inputs that are beneficial or dampen inputs that are not.

The behavior of the neural network may be modified during an iterative training process by adjusting the node/edge weights to reduce an error gradient. The computational complexity of neural network processing is a function of the number of nodes in the network. Neural networks may be sized (and/or trained) for a variety of different considerations. For example, increasing the number of nodes may improve performance and/or robustness noise rejection whereas reducing the number of nodes may reduce power consumption and/or improve latency.

Typically, machine learning algorithms are “trained” until their predicted outputs match the desired output (to within a threshold similarity). Training is broadly categorized into “offline” training and “online” training. Offline training models are trained once using a static library, whereas online training models are continuously trained on “live” data. Offline training allows for reliable training according to known data and is suitable for well-characterized behaviors. Furthermore, offline training on a single data set can be performed much faster and at a fixed power budget/training time, compared to online training via live data. However, online training may be necessary for applications that must change based on live data and/or where the training data is only partially-characterized/uncharacterized. Many implementations combine offline and online training to e.g., provide accurate initial performance that adjusts to system-specific considerations over time.

In some implementations, the NPU may be incorporated within a sensor (e.g., a camera sensor) to process data captured by the sensor. By coupling an NPU closely (on-die) with the sensor, the processing may be performed with lower power demand. In one aspect, the sensor processor may be designed as customized hardware that is dedicated to processing the data necessary to enable interpretation of relatively simple user interaction(s) to enable more elaborate gestures. In some cases, the sensor processor may be coupled to a memory that is configured to provide storage for the data captured and processed by the sensor. The sensor processing memory may be implemented as SRAM, MRAM, registers, or a combination thereof.

Other processor subsystem implementations may multiply, combine, further subdivide, augment, and/or subsume the foregoing functionalities within these or other processing elements. For example, multiple ISPs may be used to service multiple camera sensors. Similarly, neural network functionality may be subsumed with either CPU or ISP operation via software emulation.

In one embodiment, the control and data processing subsystem may be used to store data locally at the device. In one exemplary embodiment, data may be stored as non-transitory symbols (e.g., bits read from non-transitory computer-readable mediums). In one specific implementation, a memory subsystem including non-transitory computer-readable medium is physically realized as one or more physical memory chips (e.g., NAND/NOR flash) that are logically separated into memory data structures. The memory subsystem may be bifurcated into program code and/or program data. In some variants, program code and/or program data may be further organized for dedicated and/or collaborative use.

In some embodiments, the program code may be statically stored within the device as firmware. In other embodiments, the program code may be dynamically stored (and changeable) via software updates. In some such variants, software may be subsequently updated by external parties and/or the user, based on various access permissions and procedures.

### 3.1.2 Real-Time Metabolic Inference

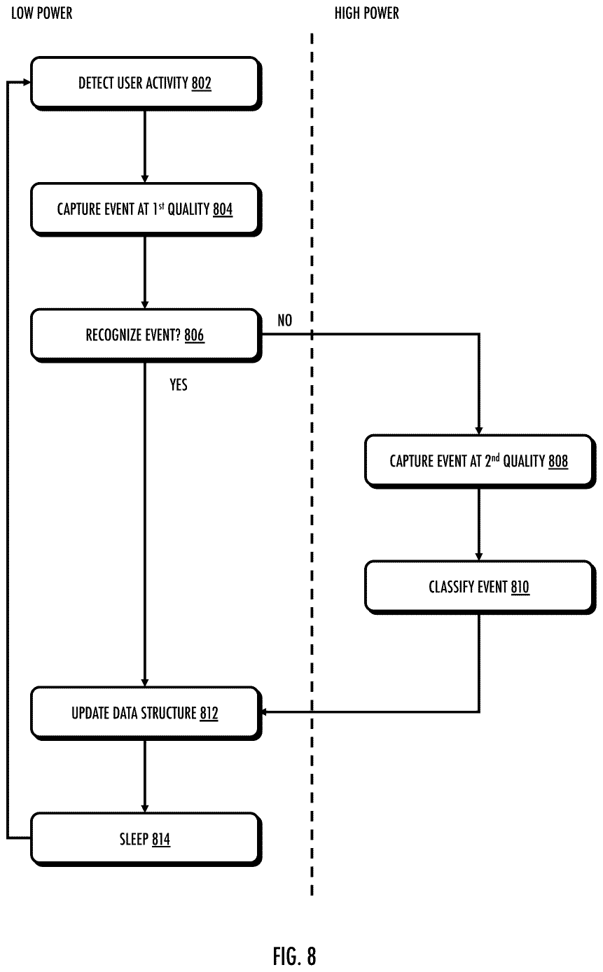

Referring now to FIG. 8, a logical flow diagram for event-driven activity recognition and adaptive data capture is shown.

At step 802, a detector such as an eye-tracking camera is configured to detect a user intent. For example, an eye-tracking camera may detect gaze vectors or saccadic movements as a user brings food up to take a bite/drink. In alternative embodiments, other sensors may serve as the activity detection subject. For example, a forward-facing camera may provide a direct observation of a user's body posture, facial movement, or locomotor pattern. A microphone may detect speech utterances or breathing cadence, a continuous glucose monitor (CGM) may detect changes in interstitial fluid indicative of activity-driven glucose uptake, and a heart-rate monitor may detect elevated beats per minute consistent with exertion. More generally, step 802 may be carried out by any user-proximal sensor that produces measurable correlates of movement, speech, physiology, gaze, etc. These various sensors collectively may be employed individually or in combination to determine whether the user is engaged in an activity (such as eating food).

Detection may include any technique for observing user actions; this may include e.g., monitoring, sensing, measuring, sampling, acquiring, etc. Monitoring applications may broadly encompass persistent, continuous observation. Sensing may be directed to a particular modality of input. Measurements may quantify a parameter of interest. Sampling techniques may collect discrete values at defined intervals. Acquisition may capture raw signal data for further processing. Artisans of ordinary skill in the related arts will readily appreciate that different implementations may stack, combine, and/or concurrently apply the concepts throughout. For example, a system may continuously monitor a microphone, sense when a signal threshold is exceeded, and then acquire a digitized audio segment for further measurement.

As used herein, the term “continuous” and its linguistic derivatives refers to ongoing observation across time, such as a camera stream that operates without interruption. In contrast, “instantaneous” and its linguistic derivatives refers observation at a specific time instant, such as a snapshot frame or a one-second audio snippet.

As used herein, the term “real-time” and its linguistic derivatives refers to systems that guarantee completion within a time constraint. For example, a system processes incoming data at the rate of acquisition, such that latency is bounded and recognition results are delivered within strict deadlines. A “near-real-time” implementation relaxes these guarantees for a specific condition (e.g., initial start-up, post-processing, etc.) permitting small but bounded delays, often acceptable in wearable or mobile contexts. A best-effort implementation imposes no strict temporal guarantees, instead delivering recognition outputs as system resources permit, such as when background processing occurs opportunistically on a low-power microcontroller. These distinctions provide flexibility for implementers to match detection timing to available hardware and energy budgets.

As previously alluded to, continuous high detail tracking is infeasible for wearable devices. However, gross tracking of user activity in monolithic chunks yields descriptive but limited information (e.g., a meal eaten, an hour of workout, etc.). Inferring metabolic impact requires a higher-order interpretation at smaller granularity: eating food at a particular portion size and rate, walking down to minute-by-minute granularity, sleep cycles/wake patterns, etc. Thus, user intent-based detection allows for detection of specific moments of interest. Focused detection can be used to trigger metabolic inference in real-time, with relatively low complexity. In other words, this granularity encompasses not only the identification of surface-level activities but also the initiation of downstream processes that relate observed events to energy expenditure, glucose utilization, cardiovascular load, or other metabolic phenomena.

At step 804, a sensing modality such as a forward-facing camera is configured to capture an event at a first quality. In one embodiment, the detector and sensing modality are distinct entities (eye-tracking camera and forward-facing camera); the detector may trigger or wake the sensing modality. In other embodiments, the detector and the sensing modality may be the same (forward-facing camera tracks hand movements bringing food up to mouth, etc.).

In one embodiment, the forward-facing camera acquires an image or video frame at a reduced resolution, a lower frame rate, or with compressed bitrate. In alternative embodiments, a microphone may capture audio at a downsampled rate, such as 8 kHz instead of 44.1 kHz, or may apply aggressive compression to limit data size. In physiological embodiments, a PPG sensor may pulse its light-emitting diode (LED) at a lower current, yielding reduced signal-to-noise ratio but conserving energy. In eye-tracking embodiments, the sampling rate of gaze points may be reduced, providing sparser data sufficient for coarse inference. Collectively, these modalities illustrate a genus of low-fidelity capture operations designed to conserve power, bandwidth, or computation while still yielding sufficient quality for recognition.

The capture operation can be continuous, instantaneous, real-time, near-real-time, or best-effort. For example, a continuous capture may involve streaming a low-resolution video, whereas an instantaneous capture may involve acquiring a single still image. A real-time capture might process low-quality audio segments as they arrive with bounded latency, while a near-real-time capture may process buffered windows of PPG data with slight delay. A best-effort capture may occur only when sufficient processor cycles are available.

Notably, the first quality may not seek to maximize fidelity but rather to balance recognition sufficiency with energy efficiency. For example, a person eating a slice of pizza and drinking a soda, is likely to take another bite of the same slice of pizza/drink of soda. In other words, a machine learning logic (discussed elsewhere) may not need high image resolution to recognize food type and assess consumed portion size, from a local library of recently consumed foods, most frequently consumed foods, etc. Lightweight image recognition can be used to update the morsel track but can trigger higher-fidelity classification (or re-classification) only as-needed. This differs from continuous high-quality recording, which may be prohibitive in mobile or wearable systems.

At step 806, recognition logic attempts to recognize the event based on the first-quality capture. In one embodiment, a lightweight neural network attempts to recognize food type and portion from low-resolution video frames. In another embodiment, a speech detection model attempts to recognize spoken activity from low-rate audio segments. In yet another embodiment, simple heuristic classifiers may detect step cadence from low-quality accelerometer or PPG signals, etc.

Recognition may be executed by artificial intelligence modules, rule-based classifiers, or template-matching algorithms. In some implementations, recognition may be performed probabilistically, outputting confidence scores rather than definitive labels. More generally, any lightweight categorization may be substituted with equal success. Examples may include computer vision classification and/or labeling.

Notably however, recognition at first quality is of a lower complexity than classification at second quality (step 810). Recognition at this stage determines whether low-fidelity evidence suffices for inference. If confidence is adequate, the system can conserve power by avoiding the higher-quality capture needed for classification. If confidence is insufficient, the system escalates fidelity at step 808. The lower-powered capture may also serve as a preliminary triage step; for example, the low-power captured image(s) of the event may be processed within onboard logic to determine whether the user is eating a morsel or if the detected activity is a false alarm.

Where necessary, the sensor escalates to a second quality setting (step 808). In one embodiment, a forward-facing camera switches from low-resolution “always-on” monitoring to high-resolution, high-frame-rate capture. Within the context of camera operation, quality may broadly encompass resolution, frame rate, bit depth/compression, exposure, sensor duty cycle, etc. For example, a first resolution morsel capture may include one or both of a low-power image (128×128, no IR illumination, long exposure) for low-power processing; whereas a second resolution morsel capture may increase resolution to 640×480 or higher and use IR illumination for proper exposure.

Other modalities may have similar analogues, for example, a microphone may increase its sampling rate to capture higher-frequency components, enabling detailed speech recognition. In physiological embodiments, a PPG sensor drives its LED at higher current and increases sample density, thereby improving signal-to-noise ratio. In gaze-tracking embodiments, the sensor may increase its sampling refresh to capture rapid saccades.

While the foregoing example is presented in the context of user intent-based adaptive escalation, the concepts may be broadly expanded to other escalation schemes. For example, adaptive escalation may be triggered periodically for calibration. Other implementations may allow external 3^{rd} parties (such as medical professionals) to adaptively escalate. Furthermore, certain types of escalations may not be “intended” by the user-certain biomarkers (e.g., heart rate, pupil dilation, etc.) may be involuntary but may reflect other aspects of user metabolism.

At step 810, the system classifies the event based on the second-quality capture. In one embodiment, a convolutional neural network classifies high-resolution images into portions and/or type of consumable. In physiological embodiments, advanced temporal models classify voice and/or heart-rate variability patterns as stress-related or exertion-related. In gaze-tracking embodiments, high-refresh data enables classification of saccadic behavior as well as other indications (e.g., pupil dilation, etc.).

In one embodiment, classification at second quality differs from recognition at first quality in that it may access more comprehensive models, including external and/or cloud-based models. For example, a user may pick up a new piece of food (e.g., dessert) which was not previously classified—this may entail accessing location data to obtain restaurant menu items and/or identification based on the likely available choices.

In one embodiment, the second-quality classification not only provides an accurate label but may also inform and update the libraries that are used for subsequent recognition at first quality. In some embodiments, classification results are fed back into lightweight models to improve their confidence, thereby reducing the need for future high-quality escalations. In other words, a user that was eating pizza and has started a slice of newly classified cake is likely to take bites of either, thus, the recognition library need only recognize those two foods to avoid the classification step in the future.

Classification at this stage may be continuous (ongoing high-quality classification), instantaneous (single burst analysis), real-time (bounded-latency decision-making), near-real-time (slight buffering delay), or best-effort (delayed labeling). The selected timing depends on the application; for example, real-time classification may be required for safety-critical gesture detection, while best-effort suffices for health-trend analysis.

At step 812, the system updates a track data structure with recognized or classified events. In one embodiment, the track is a time-ordered log that records activity labels and timestamps. In another embodiment, the track is a ring buffer storing a rolling history of recognized events. In physiological contexts, the track may include metadata such as confidence values, sampling quality, or sensor configuration. In gaze-tracking contexts, the track may store fixation sequences and dwell times.

While the foregoing discussion is presented in the context of a morsel track that is appended with morsel events as time progresses, virtually any update may be substituted with equal success. For example, subsequent analysis may revise, annotate, and/or insert new information into the morsel track. Updates may occur continuously as each new event arrives, in batched intervals, or in some combination thereof (e.g., a real-time stream that is later supplemented by better best-effort additions). The track may be stored locally on-device or uploaded to a remote service. Timing may be continuous (ongoing updates), instantaneous (single insertion), real-time (updates with guaranteed latency), near-real-time (slight delays tolerated), or best-effort (updates when system resources permit).

At step 814, the system returns one or more sensing modalities to a sleep state. In one embodiment, the forward-facing camera enters a standby mode, and the low-power eye-tracking camera remains active. In another embodiment, the microphone enters a wake-word detection state, where only minimal processing is active. In physiological embodiments, the PPG sensor powers down between sampling intervals. In gaze-tracking embodiments, the eye sensor ceases capture until reactivated by an inertial trigger.

In one embodiment, the power states may include an “off” or “sleep” state (no power), one or more low-power states, and an “on” state (full power). Transitions between power states may be described as “putting to sleep”, “waking-up”, and their various linguistic derivatives.

As a brief aside, a camera sensor's processor may include: an “off” state that is completely unpowered; a “low-power” state that enables power, clocking, and logic to check interrupts; a “on” state that enables image capture. During operation, another processor may “awaken” the camera sensor's processor. After the camera sensor's processor enters its low-power state, it services the interrupt; if a capture is necessary, then the camera sensor's processor may transition from the “low-power” state to its “on” state. Various other power management subsystems may be substituted with equal success, given the contents of the present disclosure.

While the foregoing discussion is presented in terms of “sleep”, similar techniques may include suspend, idle, standby, or hibernate. Each represents a gradation of reduced activity with different wake-latency and energy implications. For instance, standby permits rapid wake, whereas deep sleep conserves more energy but requires longer wake times. A continuous sleep policy may duty-cycle sensors at regular intervals, while an instantaneous sleep may occur immediately after a classification is completed. Real-time sleep control ensures that power transitions meet tight deadlines for wearable devices that must conserve battery without missing critical events. Near-real-time sleep control may accept slight delay in power transitions, while best-effort sleep control may opportunistically power down when resources permit.

In some embodiments, the system may perform additional steps based on the observed actions, the inferred user metabolism, or some combination thereof. For example, a processor may trigger a pharmaceutical dispenser based on the classified event and the updated track data structure. In one embodiment, if classification of the event at step 810 indicates a metabolic state requiring intervention—for example, a sustained elevation of exertion level coupled with declining glucose levels—the system actuates a connected insulin pump to deliver a pre-determined bolus. In another embodiment, if classification identifies the onset of a stress-related physiological pattern, such as elevated heart-rate variability combined with gaze fatigue, the system may actuate a dispenser to release an anxiolytic or calming agent. In still another embodiment, detection of decreased oxygen saturation or abnormal respiration may trigger a bronchodilator dispenser, such as an automated inhaler.

More generally, actuation may include dispense and/or cause administration. Here, a pharmaceutical dispensing apparatus, such as an infusion pump, patch-based transdermal delivery device, inhaler, or oral tablet dispenser is electromechanically actuated. Dispensing may be performed continuously (as in a drip infusion system), instantaneously (as in a one-time inhaler puff), in real-time (immediate bolus delivery upon threshold crossing), near-real-time (short delay to confirm event persistence), best-effort (delivery when communication with the device becomes available), or even user-actuated. Other implementations may instruct a user to take medication, or adjust their activity (e.g., stop eating, get up take a walk, manually inject insulin, etc.).

Conceptually, additional steps may be used to implement a closed therapeutic loop. Earlier steps merely detect or label user activity; these steps may interpret those labels as actionable clinical conditions. This distinction is important: recognizing that a user is walking identifies activity, but inferring that such walking, combined with current glucose data, risks hypoglycemia transforms recognition into a therapeutic trigger. Thus, some variants extend the framework from metabolic inference to direct intervention, ensuring that sensed activity leads to meaningful health outcomes.

In certain embodiments, the triggering action may be modulated by contextual parameters. For example, the pharmaceutical dispenser may be activated only if multiple modalities converge on the same classification, such as a PPG sensor indicating stress and a gaze tracker showing cognitive fatigue. In other embodiments, the dispenser may be triggered according to a dose schedule stored in the track data structure, ensuring safe administration. Safety interlocks may include verification of prior dosing, user confirmation, or physician-prescribed thresholds.

### 3.2 Aggregation

Functionally, the aggregator device aggregates user context from one or more sources (e.g., instantaneous user context (location, images, audio, etc.), accumulated user context, and/or user interest, etc.) to enable multi-modal attention for interactions between the user and other network entities. In order to do so, the aggregator device may process user context to identify attention. For example, a smartphone may run a small LLM (or similar generative intelligence logic) to encode and/or decode input (the voice commands, image, etc.) in combination with computer-vision analysis to assess attention.

Notably, conventional LLMs use a single modality (text) and assume a single user for chatbot-like functionality. In contrast, the exemplary embodiments described throughout aggregate information from multiple different modalities of data. For example, a user may use verbal commands (asking: “summarize the Wikipedia article for this.”) in relation to visual information (a gaze point that identifies an object, “this”) when interacting with multiple different network resources (e.g., a text-based LLM and a conventional webpage “Wikipedia”, etc.).

As used herein, the term “attention” refers to the inferred importance of tokens from their usage in relation to other tokens. Tokens are not limited to inputs; e.g., output tokens are also fed back, such that that transformer can attend to them as well. As previously noted, LLM transformer models assign contextual information to tokens in order to calculate scores that reflect the importance of the token relative to the other tokens. Importantly, the contextual information is dynamically inferred, and is not merely a defined weight/score for the token in isolation. Conceptually, LLMs assess both the actual meaning of words as well as their importance in a sentence, relative to the other words of the sentence. More generally, however, any mechanism that performs a dynamic assessment of contextual information, relative to other contextual information, may be considered an attention model.

In some embodiments, the aggregator may provide an additional layer of access control over the user's edge devices and/or other personal data. For example, certain network entities (e.g., a LLM) may request supplemental user context to provide better results; other embodiments may allow network entities to request user context based on scheduling and/or other trigger events. These requests may be granted, denied, and/or routed via the aggregator device. Conceptually, this may be particularly useful where combinations of different modalities of data and/or accumulated data may have more significance than isolated data points. For example, a user surfing the internet on their phone may have two separate devices (smart glasses and smart phone) which are each anonymous in isolation, yet when combined may be used by a 3^{rd} party to identify the user's identity and other sensitive information.

Furthermore, the aggregator device may also manage a user profile associated with the user and select portions of instantaneous user context to accumulate (or discard) to create accumulated user context. The user profile and accumulated user context are used to augment interactions between the user and external data sources (e.g., large language models (LLMs) as well as the broader internet). The aggregator device also manages ongoing conversation state, which may be distinct from the session state of the LLM.

Within the context of the aggregator device, the data/network interface subsystem enables communication between devices but may have additional functionality to support its aggregation functionality. For example, the aggregator device may have multiple network interfaces with different capabilities. Here, the different wireless technologies may have different capabilities in terms of bandwidth, power consumption, range, data rates (e.g., latency, throughput), error correction, etc. In one specific implementation, the aggregator device may communicate with one or more edge devices via a first network interface (e.g., a personal area network (PAN)) and the cloud service via a second network interface (e.g., a wireless local area network (WLAN)).

As a brief aside, Bluetooth is a widely used wireless protocol that is best suited for short-range communication, and data transfer between mobile devices. Bluetooth is typically used at low data transfer rates (below 2 Mbps), and often found on devices that require low power consumption. Bluetooth networks are typically small, point-to-point networks (e.g., typically <7 devices). In contrast, Wi-Fi may be configured with larger ranges (>100 m), significantly faster data rates (9.6 Gbps), and/or much larger network topologies. Wi-Fi consumes much more power and is generally used for high-bandwidth applications, etc.

Both Bluetooth and Wi-Fi use the ISM bands which are susceptible to unknown interferers; cellular connectivity often uses dedicated frequency resources (expensive), which provides significantly better performance at much lower power. Cellular modems are able to provide high throughput over very large distances (>¼ mi).

Low power network interfaces may enable a “wake-up” notification. A wake-up notification for a communication device is a signal or alert that prompts the device to transition from a low-power or sleep mode to an active state. This notification is typically used in scenarios where the device needs to conserve energy when not in use but remain responsive to incoming communications or events.

The process of a wake-up notification involves the device periodically checking for any incoming signals or messages, such as network packets or signals from other devices, while in a low-power state. When a wake-up notification is received, it triggers the device to “wake up” or transition to a fully operational state, allowing it to process the incoming data, respond to commands, or initiate actions as needed. For example, the aggregator device may receive a paging notification from the cloud service that requires information from a sleeping edge device. As another such example, an edge device that is monitoring for user interest may need to wake up the aggregator device.

As previously noted, the control and data processing logic controls the operation of a device and stores and processes data. Since the aggregator device may obtain and/or combine data from multiple sources (both edge devices and cloud services), the aggregator device may be appropriately scaled in size and/or complexity. For example, the aggregator device may have multi-core processors and/or high-level operating systems that implement multiple layers of real-time, near-real-time and/or best-effort task scheduling.

### 3.3 Resource Selection

Functionally, the cloud services are used to allocate network resources (e.g., external network entities) for processing requests by, or on behalf of, the user. For example, some (but not all) user queries may be handled with an LLM; other queries may be more efficiently handled with information gleaned from webpages and/or user databases, etc. For reasons explained in greater detail below, appropriate resource allocation improves resource utilization (e.g., computational efficiency, memory footprint, network utilization, etc.). Notably, resource selection is distinct from the other benefits of cloud operation (e.g., offloading processing, memory, and/or power consumption onto other cloud compute resources).

Cloud services refer to software services that can be provided from remote data centers. Typically, datacenters include resources, a routing infrastructure, and network interfaces. The datacenter's resource subsystem may include its servers, storage, and scheduling/load balancing logic. The routing subsystem may be composed of switches and/or routers. The network interface may be a gateway that is in communication with the broader internet. The cloud service provides an application programming interface (API) that “virtualizes” the data center's resources into discrete units of server time, memory, space, etc. During operation, a client request services that cause the cloud service to instantiate e.g., an amount of compute time on a server within a memory footprint, which is used to handle the requested service.

Referring first to the resource management subsystem, the data center has a number of physical resources (e.g., servers, storage, etc.) that can be allocated to handle service requests. Here, a server refers to a computer system or software application that provides services, resources, or data to other computers, known as clients, over a network. In most modern cloud compute implementations, servers are distinct from storage—e.g., storage refers to a memory footprint that can be allocated to a service.

Within the context of the present disclosure, data center resources may refer to the type and/or number of processing cycles of a server, memory footprint of a disk, data of a network connection, etc. For example, a server may be defined with great specificity e.g., instruction set, processor speed, cores, cache size, pipeline length, etc. Alternatively, servers may be generalized to very gross parameters (e.g., a number of processing cycles, etc.). Similarly, storage may be requested at varying levels of specificity and/or generality (e.g., size, properties, performance (latency, throughput, error rates, etc.)). In some cases, bulk storage may be treated differently than on-chip cache (e.g., L1, L2, L3, etc.).

Referring now to the routing subsystem, this subsystem connects servers to clients and/or other servers via an interconnected network of switches, routers, gateways, etc. A switch is a network device that connects devices within a single network, such as a LAN. It uses medium access control (MAC) addresses to forward data only to the intended recipient device within the network (Layer 2). A router is a network device that connects multiple networks together and directs data packets between them. Routers typically operate at the network layer (Layer 3).

The network interface may specify and/or configure the gateway operation. A gateway is a network device that acts as a bridge between different networks, enabling communication and data transfer between them. Gateways are particularly important when the networks use different protocols or architectures. While routers direct traffic within and between networks, gateways translate between different network protocols or architectures—a router that provides protocol translation or other services beyond simple routing may also be considered a gateway.

Generally, these physical resources are accessible under a variety of different configurations that are suited for different types of applications. For example, a data center might offer: infrastructure as a service (IaaS), platform as a service (PaaS), and software as a service (Saas). These classes of service provide to different levels of control/abstraction of the underlying resources. For example, IaaS might provide the most flexibility and control for cloud services, but this may require the cloud service account for and manage the underlying information technology infrastructure. In contrast, SaaS is most efficient where the client service imposes few (if any) requirements on the underlying hardware. IaaS and SaaS are at two ends of a spectrum, PaaS may provide some of the flexibility of IaaS, with some of the convenience of SaaS. As but one such example, a cloud service request for an IaaS might specify the underlying compute resource by processor, memory footprint, operating system, network setup (IP configuration), and/or application software. In contrast, a SaaS cloud service might only specify the source code for the application, etc.

Conceptually, cloud services access, reserve, and use physically remote computing resources (e.g., processing cycles, memory, data, applications, etc.) with different degrees of physical hardware and/or infrastructure management. Modern data centers handle many different cloud services from a myriad of different entities—it's not uncommon for data centers to have average utilizations north of 60% (which compares favorably to the average utilization (<1%) for dedicated servers infrastructures). Computational efficiencies are directly passed onto the cloud service as operational cost; in other words, cloud services are only charged for the resources that they request.

Cloud services are often leveraged to reduce the resource burden for embedded devices—processing intensive and/or best effort tasks can be handled in the cloud. However, efficient usage of cloud services often requires different design considerations from embedded devices. For example, cloud services benefit from careful resource allocation; over-allocation, under-allocation, and/or any other type of mis-allocation can be very inefficient (too much idle time, excessive resource churn, etc.). This is particularly problematic when scaled over multiple instances. In contrast, embedded devices are physically constrained and cannot be virtually scaled. Thus, embedded devices are often conservatively designed to match its e.g., most likely use cases, worst case use cases, etc. Embedded devices offer significant performance enhancements and/or security relative to cloud-based counterparts. For comparison, once configured, inter-data center communication is 10× slower than intra-data center communication, which is 10× slower than on-device communication.

Due to the virtualized nature of cloud services, logical entities are often described in terms of their constituent services, rather than their physical implementation.

An API (Application Programming Interface) is a set of rules and protocols that allows different software applications to communicate and interact with each other. It defines the methods, data formats, and conventions that enable access to, and functionality of, a software service, library, or platform. The illustrated implementation includes both device APIs and external APIs to interact with the other components of the system. The device APIs enable the aggregator device and/or the edge devices to communicate with the intermediary cloud service, the external APIs are used by the intermediary cloud service to launch processing requests on external network entities. In the illustrated embodiment, the external API may additionally be bifurcated into generative AI APIs, as well as more conventional internet access APIs.

An Authentication, Authorization, and Accounting (AAA) server is a system that provides authentication, authorization, and accounting services for networked resources and services. The authentication component of an AAA server verifies the identity of users or entities attempting to access a system or resource. It validates the credentials provided by the user, such as usernames, passwords, digital certificates, or other authentication factors, to ensure their authenticity.

The authorization component determines what actions or resources a user or entity is allowed to access based on their authenticated identity and specific permissions. It defines the rules and policies that govern access control and ensures that users only have access to the resources they are authorized to use.

The accounting component of an AAA server tracks and records information about the usage and consumption of network resources. It collects data related to user activities, such as the duration of sessions, data transferred, and services accessed. This data can be used for billing, auditing, network monitoring, or generating reports on resource utilization.

The AAA server manages access control to cloud resources for both the users as well as the external network resources. For example, a user may need to authenticate their identity in order to access their data. Once authenticated, authorizations and accounting are checked to ensure that the user can e.g., perform a requested action, add new data, remove data, etc. Similarly, other users and/or external network resources may need to comply with authentication and/or authorization protocols. For example, a first user may want to access the user context of a second user for group attention-based applications. Similarly, an LLM or other network entity may request supplemental user context. Depending on the user's configured access control, these requests may be granted or denied (in whole or part). In some cases, default rules may be used for convenience. Some such variants may additionally provide user notifications and/or manual override options.

A scheduling queue manages and organizes tasks or processes that are waiting to be executed. The scheduling queue determines the order in which tasks are processed, ensuring efficient utilization of resources and adherence to specific policies or priorities. Typically, a scheduling queue uses a First-In-First-Out (FIFO). The FIFO may store a collection of tasks; the addition of new tasks takes place at one end, known as the “rear” or “tail,” and the removal of elements occurs from the other end, called the “front” or “head.” More generally, any data structure suitable for job scheduling, task management, event handling, and resource allocation, may be substituted with equal success. As but one such example, round robin queues may be used to ensure that tasks are scheduled equally (or according to some fairness metric). Priority queues and Multi-level queues may be used to schedule tasks according different prioritizations and/or categorizations. Shortest Job Next (SJN) (also referred to as Shortest Job First (SJF)) and Shortest Remaining Time (SRT) queuing are often used to reduce the average wait time. Earliest Deadline First (EDF) is commonly used in time constrained applications (e.g., real-time scheduling).

A storage is configured to structure and collect data in a manner that allows efficient storage, retrieval, and manipulation of information. Here, the illustrated implementation includes user context (instantaneous user context, accumulated user context, and/or user interest), user-specific images, user-specific metadata, and user profiles.

The storage organizes user-specific data according to any number of relational schemas. Common examples of such schemas may associate data according to user, modality, time, location, metadata (extracted features, etc.). Queries may be made against the database, according to authorizations and/or other access control restrictions. For example, a user may query the database for their own data at a first level of access (unrestricted) but may have a reduced second level of access to other user's data. Other databases may be substituted with equal success.

The storage may be accessible via externalized APIs. This may enable RAG-like libraries of user-specific data (discussed elsewhere). For example, the externalized APIs may allow an external network resource to access user-specific data, according to an authorization level. In some cases, this may be extended to multiple user access (e.g., a RAG-like library for a group of users, discussed elsewhere).

An analysis engine performs analysis on metadata or input (e.g., user context and/or user interactions) to extract meaningful insights, patterns, or conclusions. The analysis engine may be configured to perform: data ingestion, pre-processing, processing, and post-processing.

During data ingestion, the analysis engine receives data or input from various sources, such as databases, files, aggregator devices and/or edge devices. If pre-processing is necessary, then the analysis engine routes data to the appropriate network component for handling and/or parses data into its relevant components. For example, edge context may be archived and/or used to update cloud context. In other examples, user input may be pre-formatted for use with e.g., an LLM-based chatbot. Some variants may additionally identify and retrieve related contextual data for initialization data.

In some variants, the analysis engine may also perform processing of the task itself. For example, some implementations may incorporate a local LLM-based chatbot. Other tasks that can be readily performed may include data retrieval, data storage, and/or other data management of the storage. Some tasks may offload processing to external 3^{rd} parties via API interfaces.

Once processing has completed, results may be presented to the user. While the disclosed embodiments describe a messaging type interface, other interfaces may be substituted with equal success. Presentation may be handled at the aggregator and/or edge devices (discussed elsewhere).

Referring back to the externalized APIs, conventional internet access APIs may be based on a server endpoint that supports one or more client endpoints. Generally, the server endpoint has a URL (which translates to an IP address) to send and receive requests and responses. Clients send and receive using HTTP methods e.g., GET, POST, PUT, DELETE. The responses are usually provided in computer-parsed formats (e.g., JSON, XML, etc.). The server and client endpoints may additionally support authentication and authorization, rate limiting, and error handling protocols. Artisans of ordinary skill in the related arts will readily appreciate that the server and client may coordinate via complementary function calls to implement very sophisticated logical interactions over the underlying API framework.

LLMs (and other generative intelligence) typically use conventional APIs to accept input text (user queries, prompts, and text passages) and provide output text (e.g., the transformed output). More recently, so-called Retrieval-Augmented Generation (RAG) LLMs have combined retrieval-based APIs with LLM functionality. A RAG-based LLM allows a client to provide a query along with a relevant library (e.g., documents or pieces of information from a predefined dataset or knowledge base). The RAG-based LLM uses the library to generate a response. In particular, a RAG-based LLM may obtain the entire library, filter/rank the library contents based on the query, and then combine the filtered documents to the LLM as contextual information to answer the query. Existing RAG-based LLMs are primarily directed to avoiding hallucinations. In other words, RAG-based LLMs are focused on providing the LLM with access to databases of pre-verified information, such that the resulting LLM output is truthful and contextually appropriate.

It will be appreciated that the various ones of the foregoing aspects of the present disclosure, or any parts or functions thereof, may be implemented using hardware, software, firmware, tangible, and non-transitory computer-readable or computer usable storage media having instructions stored thereon, or a combination thereof, and may be implemented in one or more computer systems.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

1. A method, comprising:
detecting a user activity via a first sensor;
capturing a consumption event at a first quality via a second sensor;
recognizing a food portion and a food type consumed by a user via machine-learning logic, based on the consumption event;
updating a morsel track data structure; and
sleeping the second sensor.

2. The method of claim 1, where the first sensor comprises an eye-tracking camera.

3. The method of claim 1, where the first sensor comprises an always-on forward-facing camera.

4. The method of claim 1, further comprising receiving real-time measurements of a metabolite concentration via a continuous glucose monitor.

5. The method of claim 4, correlating the morsel track data structure to the real-time measurements of the metabolite concentration.

6. The method of claim 5, further comprising notifying the user to reduce ingestion rate or initiate physical activity.

7. The method of claim 5, further comprising causing a pharmaceutical dispenser to dispense a pharmaceutical.

8. An apparatus, comprising:
a first sensor;
a machine-learning logic;
a processor; and
a non-transitory computer-readable medium comprising instructions that, when executed by the processor, cause the apparatus to:
capture a first morsel image via the first sensor;
recognize a food type and a morsel size based on the first morsel image via the machine-learning logic; and
annotate a morsel track with a morsel event comprising the food type and the morsel size.

9. The apparatus of claim 8, where the first sensor comprises a forward-facing camera configured to operate in a first mode, and where the first morsel image is captured in the first mode.

10. The apparatus of claim 9, where the forward-facing camera is further configured to operate in a second mode, and where the instructions further cause the apparatus to capture a second morsel image in the second mode.

11. The apparatus of claim 10, where the instructions further cause the apparatus to classify the food type and the morsel size based on the second morsel image.

12. The apparatus of claim 11, where the second mode consumes more power than the first mode.

13. The apparatus of claim 8, where the apparatus further comprises a network interface configured to communicate with a continuous glucose monitor.

14. The apparatus of claim 13, where the apparatus further comprises instructions that correlate the morsel track to real-time measurements of a metabolite concentration received from the continuous glucose monitor.

15. An apparatus, comprising:
a first sensor;
a second sensor;
a processor; and
a non-transitory computer-readable medium comprising instructions that, when executed by the processor, cause the apparatus to:
detect a user activity via the first sensor;
wake a second sensor to capture a morsel event;
record the morsel event in a multi-modal track; and
sleep the second sensor.

16. The apparatus of claim 15, where the first sensor comprises an eye-tracking camera and the second sensor comprises a forward-facing camera.

17. The apparatus of claim 15, further comprising a network interface configured to communicate with a continuous glucose monitor and where the instructions further cause the apparatus to record real-time measurements of a metabolite concentration within the multi-modal track.

18. The apparatus of claim 15, further comprising a network interface configured to communicate with a heart rate monitor and where the instructions further cause the apparatus to record real-time measurements of a heart rate within the multi-modal track.

19. The apparatus of claim 15, further comprising an inertial measurement unit and where the instructions further cause the apparatus to record real-time measurements of steps taken within the multi-modal track.

20. The apparatus of claim 15, further comprising a network interface configured to communicate with a sleep monitor and where the instructions further cause the apparatus to record sleep quality measurements within the multi-modal track.
